(19) United States
(12) Patent Application Publication
(10) Pub. No.: US 2004/0142450 A1
(43) Pub. Date: Jul. 22, 2004

(54) Lung epithelial cell line for propagating viruses

(76) Inventors: Sang Heui Seo, Taejon (KR); Robert C Webster, Memphis, TN (US)

Correspondence Address: DARBY & DARBY P.C., P. O. BOX 5257, NEW YORK, NY, 10150-5257, US

(21) Appl. No.: 10/477,016

(22) PCT Filed: May 10, 2002
(86) PCT No.: PCT/US02/15030

(51) Int. Cl. C12N 7/00; C12N 5/06

(52) U.S. Cl. 435/235.1; 435/325

(57) ABSTRACT

The present invention relates to a novel lung epithelial cell line, preferably a porcine lung epithelial cell line, supporting efficient high titer replication of viruses, in particular, influenza viruses. In contrast to embryonated chicken eggs and known cell culture systems for influenza replication, propagation of influenza viruses in the lung epithelial cell line of the invention is not accompanied by antigenic changes in the HA1 region of the hemagglutinin molecule and is free of contaminants which are potentially harmful for vaccine production. The cell line of the invention is useful in virus surveillance, in studies of viral pathogenesis, and in the production anti-viral vaccines for humans and animals.

This application claims priority under 35 U.S.C. § 119 (e) of U.S. Provisional Patent Application Serial No. 60/290,254, which was filed on May 10, 2001, and which is hereby incorporated herein by reference in its entirety.

The research leading to the present invention was supported, in part, by Public Health Service grants AI29680 and AI95357 and Cancer Center Support (CORE) grant CA-21765 from the National Institutes of Health. Accordingly, the U.S. government has certain rights in the invention.

### FIELD OF THE INVENTION

The present invention relates to a novel lung epithelial cell line, which supports efficient replication of viruses, particularly influenza viruses.

### BACKGROUND OF THE INVENTION

### Influenza Viruses

Influenza Virus is the most frequent cause of acute respiratory illness requiring medical intervention because it affects all age groups and because it can recur in any individual. Influenza (in particular, certain influenza A and B subtypes) is a severe cause of morbidity and mortality throughout the world, resulting in annual outbreaks in all age ranges of the population. Pandemics are the result of novel virus subtypes of influenza A, created by reassortment of the segmented genome (antigenic shift), whereas annual epidemics are the result of evolution of the surface antigens of influenza A and B virus (antigenic drift). Human influenza viruses originate from avian strains of influenza virus so that influenza infection is at its basis a zoonosis. There is evidence that pigs can serve as an intermediate host (“mixing vessel”) for the generation of new avian-originated strains that are pathogenic in humans (Scholtissek et al., Virology 1985, 147:287). The H5N1 influenza A outbreak in Hong Kong in 1997 showed that highly pathogenic influenza A viruses can also be transmitted directly from avian species to humans (Claas et al., Lancet 1998, 351:472; Suarez et al., J. Virol. 1998, 72:6678; Subbarao et al., Science 1998, 279:393; Shortridge, Vaccine 1999, 17 (Suppl. 1): S26-S29). The potential of influenza A viruses to generate new pathogenic strains from a vast number of circulating strains in the natural reservoir indicates that disease control requires monitoring these viruses and developing improved antiviral therapies and vaccines. The speed with which new strains develop demands vigilance in this monitoring effort, and stretches the capacity of current technology to produce sufficient quantities of vaccine against a newly identified pathogenic strain to prevent an epidemic or pandemic.

Influenza A, B and C, of the family Orthomyxoviridae, all have a segmented negative strand RNA genome that is replicated in the nucleus of the infected cell, has a combined coding capacity of about 13 kb, and contains the genetic information for ten viral proteins. Specifically, influenza viruses have eight negative-sense RNA (nsRNA) gene segments that encode at least 10 polypeptides, including RNA-directed RNA polymerase proteins (PB2, PB1 and PA), nucleoprotein (NP), neuramimidase (NA), hemagglutinin (association of subunits HA1 and HA2), the matrix proteins (M1 and M2) and the non-structural proteins (NS1 and NS2) (Krug et al., In The Influenza Viruses, R. M. Krug, ed., Plenum Press, New York, 1989, pp. 89-152).

Unlike positive-strand viruses (i.e., poliovirus), the negative-sense viral RNAs (vRNAs) of influenza viruses are not infectious. Only vRNA molecules encapsidated with the four viral polymerase complex proteins (PB1, PB2, PA, NP) are able to initiate a viral replication and transcription cycle. After the ribonucleoproteins (RNPs) penetrate the cell nucleus, the associated proteins begin to transcribe the nsRNAs into mRNAs and positive sense complementary RNAs [(+) cRNAs]. These cRNAs serve as templates for the synthesis of nsRNAs.

Recently developed reverse-genetics systems have allowed the manipulation of the influenza viral genome (Palese et al., Proc. Natl. Acad. Sci. USA 1996, 93:11354; Neumann and Kawaoka, Adv. Virus Res. 1999, 53:265; Neumann et al., Proc. Natl. Acad. Sci. USA 1999, 96:9345; Fodor et al., J. Virol. 1999, 73:9679). For example, it has been demonstrated that the plasmid-driven expression of eight influenza nsRNAs from a pol I promoter and the coexpression of the polymerase complex proteins result in the formation of infectious influenza A virus (Hoffmann et al., Proc. Natl. Acad. Sci. USA 2000, 97:6108).

The body of the influenza virus has a size of about 125 nm and consists of a core of nsRNAs associated with the nucleoprotein, surrounded by a viral envelope with a lipid bilayer structure. The inner layer of the viral envelope is composed predominantly of matrix proteins and the outer layer contains most of the host-derived lipid material. The so-called “surface proteins”, neuraminidase (NA) and hemagglutinin (HA), appear as spikes on the surface of the viral body.

Infectivity of novel influenza viruses depends on the cleavage of HA by specific host proteases, whereas NA is involved in the release of progeny virions from the cell surface and prevents clumping of newly formed virus. In birds, the natural hosts of influenza, the virus causes gastrointestinal infection. In mammals, replication of influenza subtypes appears restricted to respiratory epithelial cells.

The HA and NA proteins embedded in the viral envelope are the primary antigenic determinants of the influenza virus (Air et al., Structure, Function, and Genetics, 1989, 6:341-356; Wharton et al., In The Influenza Viruses, R. M. Krug, ed., Plenum Press, New York, 1989, pp. 153-174). Due to reassortment of influenza segmented genome, new HA and NA variants are constantly created for which a newly infected organism has no anamnestic immune response. Of the 15 HA and 9 NA subtypes of influenza circulating in aquatic birds, three, H1N1, H2N2, and H3N2 subtypes are known to have caused pandemics in humans (Webster et al., Microbiol. Rev. 1992, 56:152).

HA glycoprotein is the major antigen for neutralizing antibodies and is involved in the binding of virus particles to receptors on host cells. HA is synthesized in infected cells as a single polypeptide. Post-translational protease cleavage of the precursor HA results in the formation of the two subunits, HA1 and HA2, joined by a disulfide bond. Cleavage is essential for production of infectious viruses: virions containing uncleaved HA are noninfectious. The cleavage process can occur intracellularly or extracellularly. While HAs of infectious viruses are cleaved by extracellular proteases, such as from intestinal bacteria or the pancreas in vivo, the HAs of human, porcine and most avian influenza virus strains cannot be cleaved by intracellular proteases. Therefore, replication of these viruses in many cell cultures requires the addition of a protease (such as trypsin) to the maintenance medium to ensure HA cleavage, thereby permitting activation of the progeny virus so that the rounds of infection can continue (see below).

### Influenza Vaccines

The influenza vaccines currently licensed by public health authorities for use in the United States and Europe are inactivated influenza vaccines. The viruses presenting epidemiologically important influenza A and influenza B strains are grown in embryonated chicken eggs and the virus particles are subsequently purified and inactivated by chemical means. Each year the WHO selects subtypes which most likely will circulate: currently two strains for influenza A (H1N1) and (H3N2), and a B strain.

Although influenza vaccines have been in use since the early 1940's for human vaccination and since the late 1960's for equine vaccination, the existence of an extensive animal reservoir, combined with the threat of emergence of a novel influenza virus capable of causing a pandemic, has spurred research into novel therapies with which to fight the virus. Several important advances in the field of influenza have occurred in the last few years (reviewed in Cox and Subbarao, Lancet 1999, 354:1277-82). For example, an experimental live, attenuated, intranasally administered trivalent influenza vaccine was shown to be highly effective in protecting young children against influenza A H3N2 and influenza B. New antiviral drugs such as amantadine HCl and drugs based on the structure of the NA molecule were assessed in clinical trials and found to be effective against influenza A and B viruses. Other approaches to improve the efficacy of the current (killed) influenza virus vaccines include the generation of cold-adapted and genetically engineered influenza viruses containing specific attenuating mutations (reviewed in Palese et al., J. Infect. Dis., 1997, 176 Suppl 1:S45-9). It is hoped that these genetically altered viruses, in which the HA and NA genes from circulating strains have been incorporated by reassortment, can be used as safe live influenza virus vaccines to induce a long-lasting protective immune response in humans. In addition, genetically engineered influenza viruses may provide a means for expressing foreign antigens.

Currently employed influenza vaccines are based either on live virus or “killed” or “inactivated” virus. Most of the commercially available inactivated influenza vaccines are so-called “split vaccines” or “subunit vaccines”. “Split vaccines” are prepared by the treatment of the whole influenza virus with solubilizing concentrations of detergents and subsequent removal of the detergent and of the bulk of the viral lipid material. “Subunit vaccines” against influenza unlike “split vaccines” do not contain all viral proteins. Instead, “subunit vaccines” are enriched in surface proteins responsible for eliciting the desired virus neutralizing (hence protecting) antibodies upon vaccination.

Cold-adapted (ca) reassortment (CR) viruses containing the six internal genes of live, attenuated influenza A/Ann Arbor/6/60 (H2N2) or B/Ann Arbor/1/66, and the HA and NA of contemporary wild-type influenza viruses appear to be reliably attenuated. Live attenuated influenza virus vaccines administered intranasally induce local, mucosal, cell-mediated and humoral immunity. Although these vaccines appear to be efficacious in children and young adults, they may be too attenuated to stimulate an ideal immune response in elderly people, the major group of the 20000-40000 individuals in the USA dying each year as a result of influenza infection.

### Systems for Influenza Vaccine Production

For the past several decades, fertilized chicken eggs have been used as a host system to replicate human influenza viruses with infectivity titers sufficient for use in vaccine production. Clinical isolates of human influenza virus are taken from infected patients and are reassorted in embryonated chicken eggs with laboratory-adapted master strains of high growth donor viruses. The purpose of this reassortment is to increase the yield of candidate vaccine strains achieved by recombining at least the HA and NA genes from the primary clinical isolates, with the internal genes of the master strain donor viruses. This provides high growth reassortants having antigenic determinants similar to those of the clinical isolates (Robertson et al., Biologicals, 1992, 20:213-220). The reassorted influenza virus is then grown in embryonated chicken eggs, purified from virus-containing allantoic fluid of the eggs and subsequently inactivated and standardized for use as vaccines.

It is becoming widely recognized, however, that the egg-derived production of influenza virus for vaccine purposes has several important disadvantages. Thus, for the production of a safe and effective vaccine it is important that the selected vaccine strains are closely related to the circulating strains, thereby ensuring that the antibodies in the vaccinated population are able to neutralize the antigenetically similar virus. However, because of the frequency of viral mutation in antigenic sites of HA, during replication in the chicken eggs, even a single passage of a human influenza virus isolate or reassortant in chicken eggs leads to the selection of viral variants that differ in their antigenic determinants from those of the original clinical isolates (Katz et al., Virology, 1987, 156:386-395; Robertson et al., Virology, 1985, 143:166-174; Schlid et al., Nature, 1983, 303:706-709). In addition, passaging of mammalian influenza viruses in eggs can result in a change in the receptor specificity from the mammalian α-2,6-galactose oligosaccharide to the avian α-2,3-sialic acid linkage (Ito et al., J. Virol., 1997, 71:3357-3362). As a result of these events, the cultivation of influenza A and B viruses in chicken eggs often leads to the selection of viral variants, which are ineffective or significantly less effective when used in an influenza vaccine (Kodihalli et al., J Virol., 1995, 69:4888-4897; Gubareva et al., Virol., 1994, 199:89-97; Katz and Webster, J. Infect. Dis., 1989, 160:191-198; Wood et al., Virol., 1989, 171:214-221; Katz et al., Virology, 1987, 156:386-395; Robertson et al., Virology, 1985, 143:166-174).

Moreover, embryonated chicken eggs have potentially serious limitations as a host system due to (i) the varying (micro)biological quality of the eggs because of the presence of adventitious agents (e.g., other viruses); (ii) the lack of reliable year-around supplies of high-quality eggs (e.g., caused by the low susceptibility of summer eggs to influenza virus infection [Monto et al., J. Clin. Microb., 1981, 13:233-235]); (iii) the lack of production flexibility if suddenly demand increases, i.e., in case of a serious epidemic or pandemic, because of the logistic problems due to non-availability of large quantities of suitable eggs; (iv) known hypersensitivity to chicken and/or egg proteins in many subjects.

A solution for these problems may reside in tissue culture production of influenza virus. Such production method has many advantages: (i) tissue culture cell lines are available in well-defined cell bank systems free of microbiological contaminants, whereby the batch-to-batch consistency is greatly improved and a product of higher quality is obtained; (ii) use of tissue culture-based virus production increases the ability to have sufficient vaccine available in case of a serious epidemic or pandemic threat; (iii) the resulting influenza virus material is better suited for alternative routes of administration (oral, nasal, inhaled); (iv) the yearly vaccine composition recommendation can be pushed back from mid-February to mid-March, improving the efficiency of identifying the circulating pathogenic strains and creating appropriate vaccines.

Various cultured mammalian cells have been used for virus replication. These cells have been classified into at least two distinct groups. Primary diploid cells are those derived from intact tissue and have not been subcultivated. Continuous cell lines (CCLs) are cultured primary cells that replicate indefinitely and may be capable of growth in suspension culture (Haylick, In Continuous Cell Lines as Substrates for Biologicals, Arlington, Va., p. 2, 1988).

At present, many viral vaccines other than influenza are produced using primary trypsinized cells, including cells from monkey, rabbit, and hamster kidneys. Primary diploid cell cultures have certain beneficial properties such as easy preparation using simple media and bovine sera and sensitivity to a wide-range multiple viruses. However, primary diploid cells currently in use suffer from disadvantages, such as contamination by various adventitious agents, variable quality of the cells in the cell culture, different sensitivities of the cells to variants of the same virus, low virus titers and the high cost and difficulties in obtaining and preparing such cell cultures. For example, influenza production was attained in primary diploid cell cultures derived from human adenoid, rhesus monkey kidneys (Endo et al., J. Virol., 1996, 70:2055-2058; Lennette et al., Diagnostic procedures for viral and rickettsial infections, 1969, 4^{th} ed. American Public Health Association Inc., New York), and mink lung cells (Mv1Lu [ATCC Accession No. CCL-64]; see, e.g., Schultz-Cherry et al., J. Clin. Microbiol., 1998, 36:3718-20). Importantly, however, monkey kidney cells obtained from wild animals usually contain endogenous viruses (Grachev, In Guidance for the Production of Vaccines and Sera., Burgasov, ed., Medicine, Moscow, p 176, 1978; Grachev, Zh. Microbiol. Epidemiol. Immunobiol., 1987, 2:76), while mink lung cells are highly unstable in culture (Schultz-Cherry et al, 1998. J. Clin. Microbiol. 36:3718-3720). Influenza virus propagation has been also demonstrated in primary cell cultures of Chicken Embryo Fibroblasts (CEF), however, viral titers produced in these cultures are very low (Maassab, Proc. Natl. Acad. Sci. USA, 1959, 45:1035-1039).

Compared to the primary cell cultures, the advantages of using continuous cell lines are their retention of original antigenic characteristics of the infected virus, easy standardization, high susceptibility to variants of the same virus, and ability to be grown as a large mass of cells using microcarrier or suspension fermentor systems (Mizrahi, ed., Viral Vaccines, Wiley-Liss, New York, 1990, pp. 39-67; Katz et al., Virology, 1988, 165:446-456; Robertson et al., Virology, 1990, 179:3540; Katz et al., J. Infect. Dis., 1989, 160:191-198; Wood et al., Virology, 1989, 171:214-221).

Despite numerous efforts, the only presently developed continuous cell line which appears to produce sufficiently high titers of influenza virus is the MDCK cell line (Frank et al., J. Clin. Microb., 1979, 10:32-36; Schepetink and Kok, J. Virol. Methods, 1993, 42:241-250; Tobita et al., Med. Microbiol. Immunol., 1975, 162:9-14; Klenk et al., Virology, 1975, 68:426-439; Reina et al., 1997, supra; Schepetiuk et al., J. Virol. Methods, 1993, 42:241-250; Govorkova et al., J. Virol, 1996, 70:5519-5524; Govorkova et al., Virology, 1999, 262:31-38). However, this cell line has been found to produce tumors in nude mice and has thus not been certified for vaccine production.

Two other potentially useful continuous cell lines, African green monkey kidney cells (Vero; see, e.g., U.S. Pat. No. 5,824,536) and baby hamster kidney cells (BHK-21), although approved and certified by the World Health Organization (WHO) for production of human vaccines, produce titers of influenza A viruses that are significantly lower than titers obtained in MDCK cells (Govorkova et al., 1996, supra; Valette et al., Antimicrobiol. Agent and Chemotherapy, 1993, 37:2239-2240). Furthermore, the growth of influenza B in Vero cells appears to be greatly restricted as compared to MDCK cells (Nakamura et al., J. Gen. Virol., 1981, 56:199-202), while influenza growth in BHK cells, like that in eggs, results in the selection of receptor-binding variants (Govorkova et al., Virology, 1999, 262;31-38). Another potentially useful cell line, MRC-5, has extremely stringent growth media requirements making it suboptimal for large-scale production of influenza viruses for use in vaccines.

As follows from the data summarized above (see also U.S. Pat. Nos. 4,500,513; 5,824,536; 5,948,410), currently no mammalian cell line is licensed for use in the production of human influenza vaccine and it has not yet been possible to achieve high enough titer of influenza virus production in the absence of potentially harmful extraneous genetic material either in primary cell cultures or in continuous cell lines. The difficulty in attaining high influenza titers in culture is at least in part due to a “one-step growth cycle”; that is, the ability of only the originally infected cells to replicate viruses (see, e.g., Davis et al., Microbiology, Harper and Row Publishers, Ch. 44, pp. 1138-39, 1968; Endo et al., J. Virol., 1996, 70:2055-2058; Frank et al., J. Clin. Microbiol., 1979, 10:32-36; Govorkova et al., J. Virol., 1996, 70:5519-5524; Lau and Scholtissek, Virology, 1995, 212:225-2318; Reina et al., J. Clin. Microbiol., 1997, 35:1900-1901). Since the viruses of the originally infected cells are unable to infect successive numbers of cells in the same cell culture, the resulting yields are far too low to be useful in the preparation of virus vaccines.

Another significant obstacle to successful influenza virus production in tissue culture is the cytopathic effect of influenza virus. Thus, it was reported that influenza infection of epithelial cells such as MDCK or HeLa cells leads to programmed cell death (apoptosis) (Hinshaw et al., J. Virol, 1994, 68:3667-3673, Takizawa et al., J. Gen. Virol., 1993, 74:2347-2355). However, it is not yet clear whether infection of other cells or tissues by influenza viruses (e.g., epithelial cells of respiratory tracts) results in apoptosis or necrosis.

Taken together, there is a long-felt need in the art for a method of influenza virus and vaccine production in a host cell system that improves on the use of chicken eggs and existing cell culture systems. Specifically, there is a great need in the art to develop an alternative cell line that (i) supports the high titer replication of all subtypes of influenza viruses, (ii) maintains antigenic properties of the clinical isolates of the natural virus, and (iii) avoids having significant adventitious agents present in final viral preparations, which are unsuitable for vaccine production.

The present invention addresses these and other needs in the art by providing a novel continuous epithelial cell line derived from lung tissue, e.g., porcine lung epithelium. As disclosed below, the porcine lung epithelial cell line of the present invention supports replication of all tested avian, swine, equine, and human isolates of influenza A viruses as well as all tested human isolates of influenza B viruses and is useful not only for the production of influenza vaccine, but also for production of various other viruses as well as in viral surveillance and in the study of viral pathogenesis, factors that influence interspecies transmission, and virus-induced cell damage.

### SUMMARY OF THE INVENTION

The present invention addresses these and other needs in the art by providing an epithelial cell line, particularly a lung epithelial cell line, and more particularly a porcine lung epithelial cell line, that has acceptable virus production characteristics. In particular, this cell line (i) supports the high titer replication of all subtypes of influenza viruses, (ii) maintains antigenic properties of the clinical isolates of the natural virus, and (iii) avoids having significant adventitious agents present in final viral preparations, which are unsuitable for vaccine production. Such a cell line preferably has the characteristics of porcine lung epithelial cell line SJPL. As disclosed herein, in contrast to embryonated chicken eggs and known cell culture systems for influenza replication, propagation of influenza viruses in an epithelial cell line of the instant invention is not accompanied by antigenic changes in the HA1 region of the hemagglutinin molecule and is free of contaminants which are potentially harmful for vaccine production.

In a specific embodiment, the porcine lung epithelial cell line of the present invention is SJPL cell line deposited with the American Type Culture Collection (ATCC), 10801 University Boulevard, Manassas, Va. 20110-2209, USA on Apr. 5, 2001, and assigned Accession No. PTA-3256.

A novel lung epithelial cell line of the invention is useful, inter alia, for studying the influenza infection and propagation, virus surveillance, isolating functional components of influenza virus, and for sensitive, fast diagnostic and therapeutic applications.

Thus, in a specific embodiment, the present invention also provides a method for the preparation of influenza virus or virus-derived antigen(s) in cell culture comprising transfecting or infecting the lung epithelial cell line of the invention with the influenza virus or its derivatives (and may further comprise isolation and purification of the propagated viral particles and/or antigens). As disclosed herein, by providing a novel lung epithelial cell line, the present invention allows for a high titer viral production, resulting in viral titers of at least 3×10⁷ plaque forming units (PFU) per ml, preferably 5×10⁷ PFU/ml.

In contrast to other cell culture systems for influenza production known in the art, the method according to the present invention advantageously results in viral preparations, which are safe and do not contain non-acceptable amounts of deleterious genetic material, and meet the requirements set by the regulatory authorities. Thus, as disclosed in a specific embodiment, influenza virus propagation in the lung epithelial cell line of the invention is not accompanied by antigenic changes in the HA1 region of the influenza hemagglutinin (HA) molecule. Accordingly, viral preparations produced according to the method disclosed herein may be applied during the production of vaccines containing diverse influenza virus strains such as the viruses typical for human influenza, porcine influenza, equine influenza, and avian influenza.

In conjunction with the methods and lung epithelial cell line, the instant invention also provides isolated viral particles, viral antigens and virus-based vaccine preparations (the latter further comprising a pharmaceutically acceptable carrier or diluent and/or adjuvant) produced in the cell line of the invention. As disclosed herein, the viral preparations of the invention can be either infectious, non-infectious, or attenuated.

The present invention further advantageously provides a method for preventing an influenza infection in an animal (e.g., human, porcine, equine, or avian) comprising administering to the animal vaccine compositions produced using the lung epithelial cell line of the invention according to the methods disclosed above.

In a specific embodiment, the invention also provides a method of screening for anti-influenza therapeutics comprising administering a candidate agent to the influenza-producing lung epithelial cell line of the invention and testing for change in the level of influenza replication or influenza-associated protein expression compared to the control untreated cell line.

As provided in a separate embodiment, influenza virus infection of porcine lung epithelial cells does not lead to apoptosis as does infection of MDCK cells.

By providing a method for producing influenza-derived antigens in lung epithelial cells, the invention also provides a method for producing novel polyclonal antibodies to influenza-derived proteins and/or viral particles. The novel anti-influenza antibodies disclosed herein may be used diagnostically, e.g., to detect the presence and/or propagation of influenza in a cell culture or in an animal. Alternatively, these antibodies may be used therapeutically, e.g., in passive immunotherapy. In a related aspect, the invention also provides a test kit for influenza diagnostics comprising anti-influenza antibodies, influenza virus components and the lung epithelial cell line permissive for influenza replication and expressing these components.

As disclosed herein, the lung epithelial cell line of the invention can be also used for production of various viruses, viral components, and anti-viral vaccines that are unrelated to influenza. Accordingly, the scope of the present invention extends to production in the cell line of the invention of all viruses, viral components, and anti-viral vaccines, including, but not limited to, negative strand ssRNA viruses (such as Orthomyxoviridae, Paramyxoviridae, Rhabdoviridae, and Filoviridae), ambisense ssRNA viruses (such as Bunyaviridae and Arenaviridae), positive strand ssRNA viruses (such as Picornaviridae, Calciviridae, Togaviridae, Flaviviridae, Coronaviridae, and Retroviridae), dsRNA viruses (such as Reoviridae and Birnaviridae), and DNA viruses (such as Hepadnaviridae, Parvoviridae, Papovaviridae, Adenoviridae, Herpesviridae, Poxyiridae, and Iridoviridae). Specific examples of non-influenza anti-viral vaccines of the invention for human and animal use include without limitation parainfluenza vaccines, ebola virus vaccines, West Nile virus vaccines, respiratory syncitial virus vaccines, measles vaccines, mumps vaccines, yellow fever vaccines, hepatitis A and B vaccines, herpes vaccines, foot-and-mouth disease vaccines, Newcastle disease vaccines, porcine circovirus vaccines, and equine encephalitis vaccines.

The present invention meets these and other aspects of the invention, as set forth in greater detail in the Detailed Description and Examples.

### DETAILED DESCRIPTION OF THE INVENTION

The present invention advantageously provides a novel stable lung epithelial cell line, particularly a porcine lung epithelial cell line, which, upon transfection or infection, can support high titer replication of various viruses, such as (but not limited to) ambisense ssRNA viruses (e.g., Bunyaviridae and Arenaviridae), positive strand ssRNA viruses (e.g., Picornaviridae, Calciviridae, Togaviridae, Flaviviridae, Coronaviridae, and Retroviridae), dsRNA viruses (e.g., Reoviridae and Birnaviridae), DNA viruses (e.g., Hepadnaviridae, Parvoviridae, Papovaviridae, Adenoviridae, Herpesviridae, Poxyiridae, and Iridoviridae) and, in particular, negative strand RNA viruses (e.g., Orthomyxoviridae, Paramyxoviridae, Rhabdoviridae, and Filoviridae), most preferably influenza viruses.

The invention is based, in part, on the discovery of a spontaneous porcine lung epithelial cell line, and the ability of this cell line to propagate high titers of influenza virus. As disclosed herein, in contrast to embryonated chicken eggs and known cell culture systems for influenza replication, propagation of influenza viruses in the lung epithelial cell line of the invention is not accompanied by antigenic changes in the HA1 region of the hemagglutinin (HA) molecule and is free of contaminants which are potentially harmful for vaccine production. Furthermore, influenza virus infection of porcine lung epithelial cells does not lead to apoptosis, as it does in MDCK cells.

In a specific embodiment, the porcine lung epithelial cell line of the present invention is cell line SJPL deposited with the American Type Culture Collection (ATCC), 10801 University Boulevard, Manassas, Va. 20110-2209, USA on Apr. 5, 2001, and assigned Accession No. PTA-3256.

The invention is also based on a further discovery of a chicken lung epithelial cell line that has improved influenza virus replication properties compared to embryonated eggs and MDCK cells.

Further provided herein is a method for generating sufficient quantities of virions or virus-derived antigens from the lung epithelial cell line of the present invention for vaccine production. These virions or antigens can be generated for the purpose of providing a recombinant attenuated anti-virus vaccine. Preferably, as exemplified infra, the virus is influenza virus.

By providing a novel cell line supporting efficient viral replication, the present invention provides (i) in vitro cell culture model of virus propagation (useful, e.g., to identify and study factors that influence interspecies transmission and virus-induced cell damage); (ii) system for screening candidate anti-viral compounds and evaluating drug resistance; (iii) methods for diagnosing viral infection; and (iv) system for production of large quantities of virus-derived components or recombinant viral particles for antibody generation and/or vaccine development.

A novel cell line of the invention preferably has the characteristics of cell line SJPL (ATCC Accession No. PTA-3256) when one or more of its morphological, cell culture, virus propagation and production, lack of adventitious contaminants, and response to viral infection are about the same as the SJPL cell line. For purposes of the invention, having such characteristics is a qualitative, not quantitative, determination. Thus, a cell line has the influenza virus propagation characteristics of SJPL when it produces high titers of virus, and thus does not have to produce the same viral titers as described in the Examples, infra. Similarly, a cell line has a characteristic of SJPL when it does not undergo apoptosis when infected by influenza virus.

One such characteristic of a cell line of the invention is that it yields final viral preparations that are free of adventitious agents that are unsuitable for vaccine production. Such adventitious agents are contaminants, including, but not limited to, bacteria, other viruses, mycoplasma, host genome-derived fragments (e.g., encoding oncogenes), and prions.

Various health authorities set standards for acceptable vaccines, and a characteristic of a cell line of the invention is in compliance with such standards as set forth by one or more of the following public health authorities: the World Health Organization, the United States Food and Drug Administration, Public Health Agency of any State of the United States (such as, but not limited to, California, Massachusetts, and New York), the Health and Consumer Protection Directorate-General of the European Commission, Health Canada Population and Public Health Branch, Japanese Public Health Association, Australian Department of Health and Aged Care.

Taken together, specific characteristics of a cell line of the invention are that it (i) is a lung epithelial cell line; (ii) is spontaneously generated; (iii) is continuous, i.e., able to propagate indefinitely in tissue culture; (iv) is morphologically similar to SJPL; (v) produces high titers of virus; and (vi) is free of adventitious agents.

### Definitions

The term “pathogenic virus strain” is used herein to refer to any virus strain that is capable of causing disease; preferably, the virus is on the current World Health Organization (WHO), Centers for Disease Control and Prevention (CDC), or other public health authority list of likely circulating viruses. Such viruses may include (but are not limited to), e.g., members of the Orthomyxoviridae family, the Paramyxoviridae family, the Rhabdoviridae family, the Filoviridae family, the Bunyaviridae family, the Arenaviridae family, the Birnaviridae family, the Reoviridae family, the Togaviridae family, the Flaviviridae family, the Coronaviridae family, the Picornaviridae family, the Calciviridae family, the Retroviridae family, the Hepadnaviridae family, the Parvoviridae family, the Papovaviridae family, the Adenoviridae family, the Herpesviridae family, the Poxyiridae family, or the Iridoviridae family.

A “negative strand RNA virus” is a virus in which the viral genome consists of “negative strand RNA” or “nsRNA” or “vRNA”, wherein viral or virus-derived polypeptides are encoded by the “positive strand RNA” or “cRNA” which is complementary to the genome. Negative strand RNA virus families include, but are not limited to, Orthomyxoviridae, Paramyxoviridae, Rhabdoviridae, and Filoviridae. Preferably, the viral genome is from a virus that is a member of the Orthomyxoviridae virus family, and optimally has a segmented genome. Members of the Orthomyxovirdae virus family include, but are not limited to, influenza A, influenza B, influenza C, and Thogotovirus.

The term “negative strand RNA virus virions” refers to the viral particles (produced using, e.g., host cells of the invention) containing vRNA and viral proteins, which when first produced are fully infectious.

The term “influenza virus” is used herein to define a viral species of which pathogenic strains cause the disease known as influenza or flu.

As used herein, the term “viral RNA”, which includes influeza RNA, refers to RNA from the viral genome, fragments thereof, transcripts thereof, and mutant sequences derived therefrom.

A “viral gene segment” is a cDNA corresponding to a genomic RNA molecule from a segmented (discontinuous) negative strand RNA virus genome.

As used herein, “purified influenza virions” refers to a preparation of influenza viral or virus-like particles that have been isolated from the cellular constituents with which the virus normally associates, and from other types of viruses that may be present in the infected tissue. The techniques for isolating viruses are known to those of skill in the art, and include, for example, centrifugation and affinity chromatography.

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), in either single stranded form, or a double-stranded form. Double stranded DNA-DNA, DNA-RNA and RNA-RNA duplexes are possible. The term nucleic acid molecule, and in particular DNA or RNA molecule, refers only to the primary and secondary structure of the molecule, and does not limit it to any particular tertiary forms. In discussing the structure of particular nucleic acid molecules, sequences or regions may be described herein according to the normal convention of giving only the sequence in the 5′ to 3′ direction. A “recombinant nucleic acid molecule” is a nucleic acid molecule that has undergone a molecular biological manipulation.

A “coding sequence” or a sequence “encoding” an expression product, such as a RNA, polypeptide, protein, or enzyme, is a nucleotide sequence that, when expressed, results in the production of that RNA, polypeptide, protein, or enzyme, i.e., the nucleotide sequence encodes an amino acid sequence for that polypeptide, protein or enzyme. In the present invention, translation of influenza-derived (+) strand RNA yields functional viral proteins.

The term “viral gene” means a DNA or RNA sequence that codes for or corresponds to a particular sequence of amino acids which comprise all or part of one or more viral proteins or enzymes.

The terms “express” and “expression” mean allowing or causing the information in a gene or nucleic acid sequence to become manifest, for example producing an RNA or protein by activating the cellular functions involved in transcription and translation of a corresponding gene or nucleic acid sequence. A nucleic acid sequence is expressed in or by a cell to form an “expression product” such as mRNA or a protein. The expression product itself, e.g. the resulting protein, may also be said to be “expressed” by the cell.

The term “transfection” means the introduction of a foreign nucleic acid into a cell so that the host cell will express the introduced gene or sequence to produce a desired polypeptide, coded by the introduced gene or sequence. The introduced gene or sequence may also be called a “cloned” or “foreign” gene or sequence, may include regulatory or control sequences, such as start, stop, promoter, signal, secretion, or other sequences used by a cell's genetic machinery. The gene or sequence may include nonfunctional sequences or sequences with no known function. A host cell that receives and expresses introduced DNA or RNA has been “transformed” and is a “transformant” or a “clone.” The DNA or RNA introduced to a host cell can come from any source, including cells of the same genus or species as the host cell, or cells of a different genus or species.

The terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence. Plasmids are preferred vectors of the invention.

The term “polypeptide” refers to a polymer of amino acids and does not refer to a specific length of the product; thus, peptides, oligopeptides, and proteins are included within the definition of polypeptide. This term also does not refer to, or exclude, post-translational modifications of the polypeptide, for example, glycosylations, acetylations, phosphorylations, and the like.

The term “chimeric” is used herein in its usual sense: a construct or protein or virus resulting from the combination of genes from two or more different sources, in which the different parts of the chimera function together. The genes are fused, where necessary in-frame, in a single genetic construct. As used herein, the term “chimeric” refers specifically to recombinant influenza-derived nucleic acids or proteins or virions, e.g., as described by Hoffmann et al. (Proc. Natl. Acad. Sci. USA 2000, 97:6108).

As used herein, “infectious” refers to the ability of a virus to replicate in a cell and produce viral particles. Infectivity can be evaluated either by detecting virus, i.e., viral load, or by observing disease progression in the animal. Influenza virus (viral load) can be detected by the presence of viral negative strand RNAs and/or positive strand replication intermediates, e.g., detected by RT-PCR or direct hybridization techniques. It can also be detected, if present in sufficient amount, by the presence of replicon-derived proteins, e.g., detected by immunoassay or biochemical techniques. In another alternative, a culture medium isolated from a cell line supporting viral replication or extracts/samples from an animal are used to infect naive cells in culture. The important aspects of the determination of viral infectivity in vivo (i.e., in infected subjects) is the development of either an acute or chronic viral infection, which, in turn, may include either overt pathology or merely replication and propagation of the virus.

The term “viral load” or “viral titer” is used herein to refer to a quantitative amount of virus in a cell culture or in an infected animal. The term “titer” can be also used to refer to a quantitative amount of virus-derived replicons produced within a susceptible cell. A cell line is considered to produce “high titers of virus” when, upon infection with virus or transduction with a viral replication system, it produces more infectious virions than MDCK cells as determined by number of particles, number of plaque-forming units (PFU), tissue culture infectious dose TCID₅₀ or log₁₀ TCID₅₀ value (with or without trypsin treatment), or other quantitation of virion production. In a specific embodiment, a high viral titer means greater than 5.0 log₁₀ TCID₅₀, preferably greater than 5.5 log₁₀ TCID₅₀, and more preferably greater than 6.0 log₁₀ TCID₅₀. In another specific embodiment, a high titer refers to value shown in Table 1, 2, or 3 infra.

“Disease progression” refers to the course of disease or symptoms of an infected animal, and may include acute or chronic disease symptoms. “Pathothogenesis” is a particular indication of a disease progression, and refers to the pathogenic effects of viral infection, including morbidity and mortality.

An “individual” or “subject” or “animal”, as used herein, refers to vertebrates that support a negative strand RNA virus infection, specifically influenza virus infection, including, but not limited to, birds (such as water fowl and chickens) and members of the mammalian species, such as canine, feline, rodent (racine, murine, lupine, etc.), equine, bovine, ovine, caprine, porcine species, and primates, the latter including humans.

As used herein, a “biological sample” refers to a sample of tissue or fluid isolated from an individual, including but not limited to, for example, plasma, serum, spinal fluid, lymph fluid, the external sections of the skin, respiratory, intestinal and genitourinary tracts, tears, saliva, milk, blood cells, tumors, organs, and also samples of in vitro cell culture constituents (including, but not limited to, conditioned medium resulting from the growth of cells in cell culture medium, putatively viral infected cells, recombinant cells, and cell components).

As used herein, an “in vitro cell system” or an “extracorporeal cell system” refers to cells which are replicated outside of the body, i.e., cell systems not found in nature; as such, the term includes primary cultures and cell lines.

“Primary cultures”, as used herein, refers to a culture of cells that is directly derived from cells or tissues from an individual, as well as cells derived by passage from these cells, but not to immortalized cells.

As used herein, “cell line” refers to a population of cells capable of continuous or prolonged growth and division in vitro. The term “cell line” also includes immortalized cells.

The term “host cell” means any cell of any organism that is selected, modified, transformed, grown, or used or manipulated in any way, for the production of wild-type or recombinant negative strand RNA virions, or virus-derived polypeptides. A preferred host cell of the present invention is a porcine cell lung epithelial cell, specifically SJPL cell. Other exemplary host cells include chicken lung epithelial cells and lung epithelial cells from other species. Preferably the host is not a mink lung epithelial cell, especially one that produces poor viral titers.

The term “transfection” means the introduction of a foreign nucleic acid into a cell. The introduced gene or sequence may also be called a “cloned” or “foreign” gene or sequence, may include regulatory or control sequences, such as start, stop, promoter, signal, secretion, or other sequences used by a cellular molecular machinery. A host cell that receives and expresses introduced DNA or RNA has been “transfected” and is a “transfectant” or a “recombinant cell”. According to the present invention, if a transfection of influenza-derived nucleic acid results in its subgenomic replication and influenza-derived protein expression, the transfection is termed “productive”.

The term “antibody”, as used herein, includes both monoclonal and polyclonal antibodies. Additionally, single polypeptide chain antigen-binding proteins, see U.S. Pat. No. 4,946,778, are included within the term “antibody”.

A “protective immunological response” comprises a humoral (antibody) or cellular component, or both, effective to eliminate virions and infected cells in an immunized (vaccinated) subject. Thus, a protective immune response can prevent or resolve a negative strand RNA virus, e.g., influenza virus, infection. Preferably, the antigens are “surface antigens”, i.e., expressed on the surface of the virion or the surface of infected cells. More preferably, the surface antigens are glycoproteins. For influenza, the primary glycoprotein antigens are hemagluttinin (HA) and neuraminidase (NA).

As used herein, the term “immunogenic” means that the polypeptide is capable of eliciting a humoral or cellular immune response, and preferably both. An immunogenic entity is also antigenic. An immunogenic composition is a composition that elicits a humoral or cellular immune response, or both, when administered to an animal. A molecule is “antigenic” when it is capable of specifically interacting with an antigen recognition molecule of the immune system, such as an immunoglobulin (antibody) or T cell antigen receptor. An antigenic polypeptide contains an “epitope” of at least about five, and preferably at least about 10, amino acids. An antigenic portion of a polypeptide, also called herein the “epitope”, can be that portion that is immunodominant for antibody or T cell receptor recognition, or it can be a portion used to generate an antibody to the molecule by conjugating the antigenic portion to a carrier polypeptide for immunization. A molecule that is antigenic need not be itself immunogenic, i.e., capable of eliciting an immune response without a carrier.

As used herein, “a virus-specific vaccine” is a composition that can elicit protective immunity to a virus when administered to a subject. The term “vaccine” refers to a composition containing virus, inactivated virus, attenuated virus, split virus, or viral protein, i.e., a surface antigen, that can be used to elicit protective immunity in a recipient (see Furminger, In: Nicholson, Webster and Hay (eds.), Textbook of Influenza, Chapter 24, pp. 324-332, particularly pp. 328-329). It should be noted that to be effective, a vaccine of the invention can elicit immunity in a portion of the population, as some individuals may fail to mount a robust or protective immune response, or, in some cases, any immune response. This inability may stem from the individual's genetic background or because of an immunodeficiency condition (either acquired or congenital) or immunosuppression (e.g., treatment with immunosuppressive drugs to prevent organ rejection or suppress an autoimmune condition). Efficacy of anti-viral vaccines of the invention can be established in animal models.

A “protective dose” of a vaccine is an amount, alone or in conjunction with an adjuvant, effective to elicit a protective immune response in a recipient subject. Protection can also depend on the route of administration, e.g., intramuscular (preferred for an inactivated vaccine) or intranasal (preferred for an attenuated vaccine).

The term “therapeutically effective dose or amount” refers to that amount of a compound or compositions that is sufficient to result in a desired activity.

The phrase “pharmaceutically acceptable”, whether used in connection with the pharmaceutical compositions of the invention or vaccine compositions of the invention, refers to molecular entities and compositions that are physiologically tolerable and do not typically produce untoward reactions when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18^{th} Edition.

As used herein, the term “isolated” means that the referenced material is removed from its native environment, e.g., a cell. Thus, an isolated biological material can be free of some or all cellular components, i.e., components of the cells in which the native material occurs naturally (e.g., cytoplasmic or membrane component). A material shall be deemed isolated if it is present in a cell extract or supernatant. In the case of nucleic acid molecules, an isolated nucleic acid includes a PCR product, an isolated mRNA, a cDNA, or a restriction fragment. In another embodiment, an isolated nucleic acid is preferably excised from the chromosome in which it may be found, and more preferably is no longer joined or proximal to non-coding regions (but may be joined to its native regulatory regions or portions thereof), or to other genes, located upstream or downstream of the gene contained by the isolated nucleic acid molecule when found in the chromosome. In yet another embodiment, the isolated nucleic acid lacks one or more introns. Isolated nucleic acid molecules include sequences inserted into plasmids, cosmids, artificial chromosomes, and the like, i.e., when it forms part of a chimeric recombinant nucleic acid construct. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated organelle, cell, or tissue is removed from the anatomical site in which it is found in an organism. An isolated material may be, but need not be, purified.

The term “purified” as used herein refers to material that has been isolated under conditions that reduce or eliminate the presence of unrelated materials, i.e., contaminants, including native materials from which the material is obtained. For example, a purified virion is preferably substantially free of host cell or culture components, including tissue culture or egg proteins, non-specific pathogens, and the like. As used herein, the term “substantially free” is used operationally, in the context of analytical testing of the material. Preferably, purified material substantially free of contaminants is at least 50% pure; more preferably, at least 90% pure, and more preferably still at least 99% pure. Purity can be evaluated by chromatography, gel electrophoresis, immunoassay, composition analysis, biological assay, and other methods known in the art.

Methods for purification are well-known in the art. Viral particles can be purified by ultrafiltration or ultracentrifugation, preferably continuous centrifugation (see Furminger, supra). Other purification methods are possible and contemplated herein. A purified material may contain less than about 50%, preferably less than about 75%, and most preferably less than about 90%, of the cellular components, media, proteins, or other nondesirable components or impurities (as context requires), with which it was originally associated. The term “substantially pure” indicates the highest degree of purity which can be achieved using conventional purification techniques known in the art.

In a specific embodiment, the term “about” or “approximately” means within a statistically meaningful range of a value. Such a range can be within an order of magnitude, preferably within 50%, more preferably within 20%, more preferably still within 10%, and even more preferably within 5% of a given value or range. The allowable variation encompassed by the term “about” or “approximately” depends on the particular system under study, and can be readily appreciated by one of ordinary skill in the art.

### General Techniques

In accordance with the present invention there may be employed conventional molecular biology, microbiology, biochemistry, genetics and immunology techniques within the skill of the art for the production of recombinant nucleic acids, influenza-producing cell cultures, infectious viral particles, viral and recombinant proteins, antibodies, etc. Such techniques are explained fully in the literature. See, e.g., Sambrook, Fritsch and Maniatis, Molecular Cloning: A Laboratory Manual, Second Edition, 1989, Cold Spring Harbor Laboratory Press (herein “Sambrook et al., 1989”); DNA Cloning: A Practical Approach, Glover ed., Volumes I and II, 1985; Oligonucleotide Synthesis, Gait ed., 1984; Nucleic Acid Hybridization, Hames and Higgins eds., 1985; Transcription And Translation, Hames and Higgins eds., 1984; Animal Cell Culture, Freshney ed., 1986; Immobilized Cells And Enzymes, IRL Press, 1986; Perbal, A Practical Guide To Molecular Cloning, 1984; Current Protocols in Molecular Biology, Ausubel et al., eds., 1994, John Wiley & Sons.

These routine techniques apply to the preparation of a cell culture of the invention, preparation (i.e., purification and isolation) of natural or recombinant virus, detection of viral replicons and antigens, isolation of viral gene segment cDNA clones, insertion of such cDNAs into plasmids, and transfection or infection of host cells with the virus. For example, routine techniques of site-directed mutagenisis or gene modification permit modification of viral genes to develop attenuated or defective virus, as set forth below, or to obtain viral proteins that incorporate novel epitopes.

### Lung Epithelial Cell Lines

The present invention, in addition to cell line SJPL deposited with the American Type Culture Collection (ATCC) on Apr. 5, 2001, and assigned Accession No. PTA-3256, provides for generating cell lines having the characteristics of SJPL. Normal lung tissue can be digested, e.g., with trypsin, to form a single cell suspension. The cells can be suspended at an appropriate cell density, i.e., a cell density that permits growth without excessive crowding, transformation, or metabolic distress; for example, about 5×10⁵ cells per ml is an appropriate cell density. The cell suspension is added to a tissue culture flask, plate, or other container, preferably coated with collagen. Tissue culture medium is selected to support cell viability and growth. In its essence, a buffered cell culture medium is an isotonic buffered aqueous solution, such as phosphate buffered saline (PBS), Tris-buffered saline, or HEPES-buffered saline. In a preferred embodiment, the medium is Dulbecco's modified Eagle's medium (DMEM). Other tissue culture media can also be used, including basal medium Eagle (with either Earle's or Hank's salts), Iscove's modified Dulbecco's medium (IMDM), Medium 199, Minimal Essential Medium (MEM), Eagle (with Earle's or Hank's salts), RPMI, Dulbecco's phosphate buffered salts, Earle's balanced salts (EBSS), Hank's medium (not containing phenol red), and Hank's Balanced Salts (HBSS). These media can be supplemented, e.g., with serum (e.g., about 5% to about 15%, preferably about 10%, fetal bovine serum or other mammalian serum, preferably free of endotoxins and other contaminants), glucose, Ham's nutrients, antibiotics, antimycotic agents, non-essential amino acids, reducing agents, HEPES, etc. If desired, serum-free medium can also be sued. Other components, such as sodium bicarbonate and L-glutamine, can be specifically included or omitted. Media, salts, and other reagents can be purchased from numerous sources, including Sigma, Gibco BRL, Life Technologies, Mediatech, and other companies.

Although a lung epithelial cell line of the invention preferably establishes itself spontaneously, it is possible to use transforming factors, such as radiation and chemicals (but preferably not viruses) to create a cell line capable of continuous growth in culture. A cell line of the invention can be cultured continuously, with cell passaging at appropriate intervals, e.g., about 7 days, preferably about 34 days. Suitable culture conditions will depend, in part, on the preferences of the cells and the nature of the culture medium, but will generally be selected for effective cell growth. Such conditions include temperature (usually about 37° C.) and atmosphere (usually humid, with about 5% to about 15% CO₂ and normal air; the CO₂ concentration is set to maintain pH for the specific culture medium and buffer characteristics). Cell passaging may involve trypsinization (or other mechanical or enzymatic treatment) to release the cells. Collagen may be omitted from the tissue culture vessel after the second or later passage.

Sources of lung tissue include, but are not limited to, human, porcine, canine, feline, avian (particularly chicken, turkey, duck, goose, and other fowl), borine, ovine, caprine, equine, rodent, mustelidae (including mink, provided however that the cell line developed has the characteristics of the SJPL cell line), etc. Thus, the invention provides lung epithelial cell lines for each of these classes, families or genera, with the proviso that the lung epithelial cell line is not a mink lung epithelial cell line unless the mink lung epithelial cell line has the characteristics of SJPL, preferably all the characteristics of SJPL.

### Virus Production

As noted above, the present invention provides an efficient and economic strategy for production of vaccines for treating or viral infections, in particular, negative strand RNA virus infections. As disclosed herein, a lung epithelial cell line of the invention provides high titers of replicated virus and/or virus-derived antigens. These viruses and/or antigens are suitable for, and included in, virus vaccines of the invention, for which the replicated virus is inactivated and/or attenuated.

In a specific embodiment, the invention thus provides replicated influenza viruses and vaccines that have substantially similar antigenicity to the viral clinical isolates, relative to chicken egg-grown viruses, where selection pressures in the eggs change the viruses' antigenicity from that of the clinical isolates, such as through mutation of the HA gene.

In contrast to influenza viruses grown in chicken eggs or presently used cell lines, replicated influenza viruses and vaccines of the present invention provide (i) substantially similar antigenicity to the clinical isolate; (ii) consistently high titers; (iii) lack of contamination by adventitious agents, and (iv) consistent cell growth qualities.

Influenza virus vaccines of the invention can include at least one replicated virus strain (e.g., 1-50 strains) of a mammalian influenza virus A or B. Alternatively, these vaccines may be derived from recombinant constructs, e.g., as described by Neumann et al. (Proc. Natl. Acad. Sci. USA 1999, 96:9345), Fodor et al. (J. Virol. 1999, 73:9679), or preferably Hoffmann et al. (Proc. Natl. Acad. Sci. USA 2000, 97:6108; U.S. Provisional Patent Application Serial No. 60/200,679, and International Application PCT/US01/13656).

Accordingly, the present invention encompasses the use of an eight-plasmid DNA transfection system for the rescue of infectious RNA virus (e.g., influenza virus) from cloned cDNA. As described by Hoffmann et al. (supra), in this plasmid-based expression system, viral cDNA is inserted between the RNA polymerase I promoter and terminator sequences. This entire polymerase I transcription unit is flanked by an RNA polymerase II promoter and a polyadenylation site. The orientation of the two transcription units allows the synthesis of negative-sense viral RNA and positive-sense mRNA from one viral cDNA template. In particular, for influenza A virus, preferred viral gene segments useful in the plasmid-based system of the invention encode viral polymerase complex proteins, M proteins, and/or NS proteins from a strain well adapted to grow in cell culture or from an attenuated strain, or both.

The eight-plasmid virus productuion system may be also used to generate single and quadruple reassortant viruses. A preferred “reassortment” virus of the invention is a virus in which gene segments encoding antigenic proteins from a pathogenic virus strain are combined with gene segments encoding viral polymerase complex or other similar genes (e.g., non-glycoprotein genes, including M genes and NS genes) from viruses adapted for growth in culture (or attenuated viruses). The reassortment virus thus carries the desired antigenic characteristics in a background that permits efficient production in a host cell. Such a reassortment virus is a desirable “virus seed” for production of virions to produce vaccine (see Furminger, supra).

For virus production, the lung epithelial cell line of the invention can be either infected with virus or transfected with virus-encoding plasmid constructs. For example, for the production of an inactivated influenza vaccine, six plasmids containing the non-glycoprotein segments from a high yield strain (e.g., PR/8/34 (H1N1) or WSN/33 (H1N1)) can be co-transfected with two expression plasmids containing the HA and NA cDNA of the recommended vaccine subtype. Since no helper virus is required, the generated virus is an influenza virus with the desired gene constellation.

As described above, techniques for the preparation of natural or recominant virus for infection, isolation of viral gene segment cDNA clones, modification of viral genes to develop attenuated or defective virus, insertion of viral gene segment cDNAs into plasmids, and transfection or infection of cells with the virus or virus-encoding plasmid vectors are well known in the art.

The methods for detection of viral replicons in a cell line of the invention are also well known in the art and include without limitation polymerase chain reaction (PCR), reverse transcriptase-polymerase chain reaction (RT-PCR), Nothern blot analysis, and detection of virus-derived antigens (e.g., by Westen blot, flow cytometry, or immunofluorescence) or virus-associated reporter genes.

Following propagation in a host cell line, newly formed virions can be isolated and purified using routine techniques. For example, viral particles can be purified from the cell culture medium by ultrafiltration or ultracentrifugation (see Furminger, supra).

As disclosed herein, replicated influenza virus of the invention, in isolated, purified or concentrated form, preferably has an infectivity titer of about 3×10⁷ plaque forming units (PFU) per ml, more preferably 5×10⁷ PFU/ml.

### Vaccines

The present invention also provides vaccine compositions comprising at least one strain of a replicated influenza virus of the present invention, in inactivated or attenuated form, optionally further comprising at least one of: (a) at least one pharmaceutically acceptable carrier or diluent; (b) at least one adjuvant and/or (c) at least one viral chemotherapeutic agent. Such carrier, diluent, adjuvant or chemotherapeutic agent enhances at least one immune response to at least one pathogenic influenza virus in an animal administered the vaccine composition.

The present invention also provides a method for eliciting an immune response to at least one influenza virus strain in an animal, which response is prophylactic or therapeutic for an influenza virus infection. The method comprises administering to the animal a vaccine composition comprising an inactivated and/or attenuated, replicated influenza virus of the present invention. The composition is provided in an amount that is protective or therapeutic for the animal against a clinical influenza virus pathology caused by infection with at least one influenza A or B virus strain.

Virus produced in accordance with the invention can be used in traditional or new approaches to vaccination (see Bilsel and Kawaoka, In: Nicholson, Webster and Hay (eds.), Textbook of Influenza, Chapter 32, pp. 422-434), particularly in the development of live, attenuated vaccines (discussed in greater detail infra).

Much effort has gone into the development of influenza vaccines (see Wood and Williams, In: Nicholson, Webster and Hay (eds.), Textbook of Influenza, Chapter 23, pp. 317-323). While much of this section relates to influenza vaccines, the scope of the present invention extends to all anti-viral vaccines, including, but not limited to, negative strand ssRNA virus vaccines (such as Orthomyxoviridae, Paramyxoviridae, Rhabdoviridae, and Filoviridae vaccines), ambisense ssRNA virus vaccines (such as Bunyaviridae and Arenaviridae vaccines), positive strand ssRNA virus vaccines (such as Picornaviridae, Calciviridae, Togaviridae, Flaviviridae, Coronaviridae, and Retroviridae vaccines), dsRNA virus vaccines (such as Reoviridae and Birnaviridae vaccines), and DNA virus vaccines (such as Hepadnaviridae, Parvoviridae, Papovaviridae, Adenoviridae, Herpesviridae, Poxyiridae, and Iridoviridae vaccines). Specific examples of non-influenza anti-viral vaccines of the invention for human and animal use include without limitation parainfluenza vaccines, ebola virus vaccines, West Nile virus vaccines, respiratory syncitial virus vaccines, measles vaccines, mumps vaccines, yellow fever vaccines, hepatitis A and B vaccines, herpes vaccines, foot-and-mouth disease vaccines, Newcastle disease vaccines, porcine circovirus vaccines, and equine encephalitis vaccines.

Three types of inactivated influenza vaccines are currently available: whole virus, split-product, and surface antigen vaccines (see Wood, In: Nicholson, Webster and Hay (eds.), Textbook of Influenza, Chapter 25, pp. 333-345). Because the present invention permits the high titer production of a desired virus, it advantageously positions a vaccine manufacturer to generate a sufficient quantity of vaccine to meet public health needs and ensure standardization, which is an important requirement currently mitigated by the need to produce clinical quantities of vaccine, usually in 8 to 9 month period (Wood, supra, p. 333).

Vaccine safety is also a concern (see Wiselka, In: Nicholson, Webster and Hay (eds.), Textbook of Influenza, Chapter 26, pp. 346-357). Importantly, in contrast to currently employed production systems, the lung epithelial cell line of the invention permits production of viral vaccines that are devoid of detectable non-influenza nucleic acids, heterologous viruses, or bacteria.

Adjuvants have been used with influenza vaccines (Wood and Williams, supra). The term “adjuvant” refers to a compound or mixture that enhances the immune response to an antigen. An adjuvant can serve as a tissue depot that slowly releases the antigen and also as a lymphoid system activator that non-specifically enhances the immune response (Hood, et al., Immunology, Second Ed., 1984, Benjamin/Cummings: Menlo Park, Calif., p. 384). Often, a primary challenge with an antigen alone, in the absence of an adjuvant, will fail to elicit a humoral or cellular immune response. Adjuvants include, but are not limited to, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, and potentially useful human adjuvants such as BCG (bacille Calmette-Guerin) and Corynebacterium parvum. An example of a preferred synthetic adjuvant is QS-21. Alternatively, or in addition, immunostimulatory proteins, as described below, can be provided as an adjuvant or to increase the immune response to a vaccine. Preferably, the adjuvant is pharmaceutically acceptable.

Vaccination effectiveness may be enhanced by co-administration of an immunostimulatory molecule (Salgaller and Lodge, J. Surg. Oncol. 1998, 68:122), such as an immunostimulatory, immunopotentiating, or pro-inflammatory cytokine, lymphokine, or chemokine with the vaccine, particularly with a vector vaccine. For example, cytokines or cytokine genes such as interleukin (IL)-1, IL-2, IL-3, IL-4, IL-12, IL-13, granulocyte-macrophage (GM)-colony stimulating factor (CSF) and other colony stimulating factors, macrophage inflammatory factor, Flt3 ligand (Lyman, Curr. Opin. Hematol., 5:192, 1998), as well as some key costimulatory molecules or their genes (e.g., B7.1, B7.2) can be used. These immunostimulatory molecules can be delivered systemically or locally as proteins or by expression of a vector that codes for expression of the molecule.

### Inactivated Influenza Vaccines

Inactivated virus vaccines are well established for vaccinating against influenza (see Nichol, In: Nicholson, Webster and Hay (eds.), Textbook of Influenza, Chapter 27, pp. 358-372). Virus can be inactivated by treatment with formaldehyde, beta-propiolactone, ether, ether with detergent (such as Tween-80), cetyl trimethyl ammonium bromide (CTAB) and Triton N101, sodium deoxycholate and tri(n-butyl) phosphate (Furminger, supra; Wood and Williams, supra). Prior to inactivation, the virions can be isolated and purified by centrifugation (Furminger, supra, see p. 326). To assess the potency of the vaccine, the single radial immunodiffusion (SRD) test can be used (Schild et al., Bull. World Health Organ.1975, 52:43-50 and 223-31; Mostow et al., J. Clin. Microbiol. 1975, 2:531). The dose needed for a satisfactory immune response has been standardized and is 15 μg HA/strain/dose. The inactivated vaccine can be administered intramuscularly by injection.

### Live Attenuated Influenza Vaccines

Attenuated cold adapted live influenza vaccines have been developed (see Keitel and Piedra, In: Nicholson, Webster and Hay (eds.), Textbook of Influenza, Chapter 28, pp. 373-390; Ghendon, In: Nicholson, Webster and Hay (eds.), Textbook of Influenza, Chapter 29, pp. 391-399). The ability to generate influenza virus using reverse genetics entirely from eight plasmids (encoding each of the six cDNAs of the internal genes [PB2, PB1, PA, NP, M, NS] and glycoproteins HA and NA) allows adjustment of the attenuation of a vaccine strain and enables development of a vaccine strain optimally suited for any target population (see, Bilsel and Kawaoka, supra; Hoffmann et al., Proc. Natl. Acad. Sci. USA 2000, 97:6108).

It is expected that the genetic modification of the coding or noncoding region of the internal genes improves the safety, infectivity, immunogenicity and protective efficacy of the vaccine, in addition to permitting development of attenuated virus.

The manipulation of the HA gene can also increase the safety of a vaccine strain. For example, removal of basic amino acids found in the connecting peptide of H5 or H7 glycoproteins of highly pathogenic avian influenza A viruses can increase the safety of the vaccine.

### Mucosal Vaccination

Mucosal vaccine strategies are particularly effective for many pathogenic viruses, since infection often occurs via the mucosa. The mucosa harbors dendritic cells, which are important targets for influenza vaccines and immunotherapy. Thus, mucosal vaccination strategies for inactivated and attenuated virus vaccines are contemplated. While the mucosa can be targeted by local delivery of a vaccine, various strategies have been employed to deliver immunogenic proteins to the mucosa.

In a specific embodiment, the vaccine can be administered in an admixture with, or as a conjugate or chimeric fusion protein with, cholera toxin, such as cholera toxin B or a cholera toxin A/B chimera (Hajishengallis, J Immunol., 154:4322-32, 1995; Jobling and Holmes, Infect Immun., 60:4915-24, 1992). Mucosal vaccines based on use of the cholera toxin B subunit have been described (Lebens and Holmgren, Dev. Biol. Stand. 82:215-27, 1994). In another embodiment, an admixture with heat labile enterotoxin (LT) can be prepared for mucosal vaccination.

Other mucosal immunization strategies include encapsulating the virus in microcapsules (U.S. Pat. No. 5,075,109, No. 5,820,883, and No. 5,853,763) and using an immunopotentiating membranous carrier (WO 98/0558). Immunogenicity of orally administered immunogens can be enhanced by using red blood cells (rbc) or rbc ghosts (U.S. Pat. No. 5,643,577), or by using blue tongue antigen (U.S. Pat. No. 5,690,938).

### Additional Diagnostic and Therapeutic Applications

By providing a method for producing influenza-derived antigens in lung epithelial cells, the invention also provides a method for producing novel polyclonal antibodies to influenza-derived proteins and/or viral particles comprising administering an immunogenic amount of influenza-derived proteins isolated from the cell cultures described above to an animal, and isolating generated anti-influenza antibodies. A further method for producing antibodies to influenza comprises screening a human antibody library for reactivity with influenza-derived proteins of the invention and selecting a clone from the library that expresses a reactive antibody.

The novel anti-influenza antibodies disclosed herein may be used diagnostically, e.g., to detect the presence and/or propagation of influenza in a cell culture or in an animal. Alternatively, these antibodies may be used therapeutically, e.g., in passive immunotherapy.

In a related aspect, the invention also provides a test kit for influenza diagnostics comprising anti-influenza antibodies, influenza virus components and a lung epithelial cell line permissive for influenza replication and expressing these components.

The present invention further advantageously provides methods for screening for agents capable of modulating influenza infection and/or replication and/or virion assembly. Such methods include administering a candidate agent to the influenza-propagating lung epithelial cell line of the invention, and testing for an increase or decrease in a level of influenza replication or influenza-associated protein expression compared to a level of influenza replication or influenza-associated protein expression in a control untreated cell line (e.g., in the same cell line prior to administration of the candidate agent), wherein a decrease in the level of influenza replication or influenza-associated protein expression is indicative of the inhibitory activity of the agent. Agent-mediated inhibition of virion formation can be detected microscopically (performed directly or after immunostaining); and changes in infectivity of generated influenza virus particles can be assayed by isolating them from the cell culture medium and applying to naive cells or a susceptible animal model.

In a specific embodiment, the influenza-replicating lung epithelial cell line of the present invention provides a convenient system for high-throughput initial screening of potential anti-influenza therapeutics. Such high-throughput screening system involves applying test compounds to the lung epithelial cell microcultures supporting influenza replication (growing, e.g., in 96- or 324-well microtiter plates) followed by measuring changes (e.g., using multi-plate readers or scanners) in influenza replication and/or influenza-associated protein expression and/or influenza infectivity. According to the instant invention, candidate therapeutic compounds include without limitation small molecule enzyme inhibitors (e.g., chelating agents), inhibitory peptides, inhibitory (e.g., transdominant-negative) proteins, antibodies, ribozymes, and antisense nucleic acids.

As disclosed herein, the anti-influenza therapeutic compounds identified using the initial in vitro screening methods of the present invention can be further characterized for their ability to affect influenza propagation using secondary screens in cell cultures and/or susceptible animal models. Based on the tropism of the influenza, a preferred animal model of the present invention is a pig, or even more preferably a minipig. Test animals will be treated with the candidate compounds that produced the strongest inhibitory effects in cell culture-based assays (control animals would not be treated, and, if available, a positive control could also be employed). A compound that protects animals from infection by the virus and/or inhibits viral propagation leading to pathogenicity, would be an attractive candidate for development of an agent for treatment/prevention of influenza infection. In addition, the animal models provide a platform for pharmacokinetic and toxicology studies.

### EXAMPLES

The following examples illustrate the invention without limiting its scope.

### Example 1

### Generation of SJPL Porcine Lung Epithelial Cell Line

A porcine lung epithelial cell line (SJPL) was spontaneously established from the normal lungs of a 4-week-old female Yorkshire pig. Briefly, approximately 5 g of normal lung tissue from a 4-week-old pig was digested with 30 ml Ix trypsin-EDTA containing 0.5 g porcine trypsin and 0.2 g Na-EDTA per liter of HBSS (Sigma Chemical Co., St. Louis, Mo.) at 37° C. 5×10⁵ cells per ml were plated on a tissue culture flask (75 cm²) which was pre-coated with 0.01% of collagen solution (Sigma) with Dulbecco's modified Eagle's medium (DMEM; Life Technologies, Gaithersburg, Md.) supplemented with 10% fetal bovine serum (FBS) (Summit Biotechnology, Ft. Collins, Colo.), 1% sodium pyruvate, 1% L-glutamine (Life Technologies, Gaithersburg, Md.), 1.4% MEM nonessential amino acids (Life Technologies), and 1% antibiotic-antimycotic solution (Sigma). Cells were passaged at an interval of 34 days using 1× trypsin-EDTA (Life Technologies) until cells were established for continuous growing. Starting from the second passage, collagen coating was not used.

SJPL cell line was continuously cultured in DMEM supplemented with 10% FBS, 1% sodium pyruvate, 1% L-glutamine, 1.4% MEM nonessential amino acids, and 1% antibiotic-antimycotic solution.

### Example 2

### Influenza Virus Production in SJPL Porcine Lung Epithelial Cell Line

### Materials and Methods

Cells. An epithelial cell line (SJPL) was spontaneously established from the normal lungs of a 4-week-old pig as described in Example 1, above. MDCK cells were received from the National Institute of Medical Research (Mill Hill, UK) and were cultivated in Eagles' minimal essential medium containing 5% heat-inactivated FBS at 37° C. in 5% CO₂. Mink lung epithelial cells (Mv1Lu [ATCC Accession No.CCL-64]) were obtained from the American Type Culture Collection (Manassas, Va.) and were cultivated in Eagles' minimal essential medium containing 1.5 g/L sodium bicarbonate, 0.1 mM nonessential amino acids, 1.0 mM sodium pyruvate, and 10% heat-inactivated FBS at 37° C. Because of their sensitivity to trypsin, Mv1Lu cells were not used to study replication efficiency. Mv1Lu cells were detached from tissue-culture flask 18 h after cells were infected with influenza viruses, even when the cells were in the presence of 0.5 μg/ml L-1-tosylamide-2-phenylethyl chloromethyl ketone (TPCK)-treated trypsin (Worthington Biochemical Corporation, Lakewood, N.J.).

Viruses. The influenza viruses used in this study were obtained from the repository at St. Jude Children's Research Hospital. All viruses were grown in 11-day-old embryonated eggs, and 10-fold serial dilutions of the viruses were titrated in eggs before the viruses were used in replication efficiency experiments. The influenza virus used in the antigenic stability study was grown in and isolated from MDCK and SJPL cells. The original virus was from a patient's saliva gargle sample in Memphis, Term., during 1999.

Virus titration. SJPL and MDCK cells (5×10⁵ per well) were seeded in six-well tissue-culture plates and allowed to grow to confluence. Cells were washed with phosphate-buffered saline (PBS, pH 7.2) twice and infected with 10 EID₅₀ (i.e., 10 times the virus dose that will infect 50% of the eggs in a population) for 1 hour at 37° C. in a humidified incubator containing 5% CO₂. Infected cells were washed twice with warm PBS (pH 7.2), and 2 ml of medium (DMEM for SW-00 and MEM for MDCK cells) containing 1 μg/ml TPCK-treated trypsin was added to each well. Supernatants containing viruses were collected 50 hours after infection for virus titration.

MDCK cells were used for titration of MDCK-grown virus, and SJPL cells were used for titration of SJPL-grown virus. Supernatants from virus-infected cells were serially diluted 10-fold in medium (DMEM for SJPL-grown virus and MEM for MDCK-grown virus) containing 0.3% bovine serum albumin (BSA) and 1 μg/ml TPCK-trypsin, and 0.1 ml of the dilutions was added to four replicate wells in a 96-well plate. Seventy-two hours after infection, the presence of virus was determined by HA with 0.5% chicken red blood cells. The virus titers were expressed in units of log₁₀ TCID₅₀/ml.

Growth Curve. Influenza viruses A/Sydney/5/97 (H3N2), B/Memphis/1/84, and A/Swine/IA/17672/88 (H1N1) were titrated in eggs by using serial 10-fold dilutions. Monolayers of SJPL and MDCK cells in a 6-well plate were infected with 10 EID₅₀ of each virus. Infected cells were washed three times with warm PBS (pH 7.2), and medium containing 0.3% BSA and 1 μg/ml TPCK-treated trypsin was added to each well. The cells were incubated at 37° C. in a 5% CO₂. At different times after infection, 50 μl of supernatant was removed. The presence of viruses in supernatant was determined by titration as described in the preceding section.

Isolation of influenza viruses from human clinical samples. Confluent monolayers of SJPL and MDCK cells in a 24-well tissue-culture plate were treated with 0.1 ml of human clinical gargle sample collected in sterile PBS, and the cells and sample were incubated together for 40 min. At 37° C. in a humidified incubator with 5% CO₂, medium containing 0.3% BSA and 1 μg/ml TPCK-treated trypsin was added to the infected cells, which were then incubated for an additional 3 days. We performed hemagglutination (HA) assays with 0.5% turkey red blood cells to determine whether influenza virus was present. Turkey red blood cells were used because recently isolated human influenza viruses do not easily agglutinate chicken red blood cells.

Nucleotide sequencing of HA1 gene segments of viruses grown in MDCK cells, in SJPL cells, and in eggs. Influenza A virus (A/Memphis/4/99) from the human clinical gargle samples was passaged three times in MDCK cells and in SJPL cells. These viruses were also adopted for growth in eggs and passaged three times. After three passages, RNA was extracted from virus in the culture supernatant of allantoic fluids with the RNeasy Mini Kit (Qiagen, Santa Clara, Calif.) and subjected to reverse transcription PCR (RT-PCR; SuperScript Preamplification System, Life Technologies, Gaithersburg, Md.). PCR products were purified with the QIAquick PCR purification kit (Qiagen) and sequenced by staff members in the Hartwell Center for Bioinformatics and Biotechnology at St. Jude Children's Research Hospital. They used BigDye Terminator Cycle Sequencing Ready Reaction Kits with AmpliTaq DNA polymerase, FS (Perkin-Elmer, Applied Biosystems, Inc., Foster City, Calif.) and synthetic olignucleotides. Analysis and translation of nucleotide sequence data were performed with the Lasergene sequence analysis software package (DNASTAR, Madison, Wis.).

Indirect immunofluorescence assay and analysis of cytopathic effect (CPE). SJPL cells were cultured to confluence in Lab-Tek chamber slides (Nalge Nunc International, Naperville, Ill.). Cells were washed with PBS (pH 7.2) and infected with an H3N2 (A/Sydney/5/97) influenza virus (multiplicity of infection [m.o.i.], 5) for 1 h at 37° C. in a humidified incubator containing 5% CO₂. Infected cells were washed twice with warm PBS (pH 7.2); DMEM containing 0.3% BSA was added to each chamber. Infected cells were incubated overnight at 37° C. in the humidified incubator containing 5% CO₂ before they were fixed with 80% cold acetone in water. The fixed cells were incubated on ice with an anti-mouse monoclonal antibody to nucleoprotein of influenza A virus for 30 min and then washed three times with cold PBS (pH 7.2) containing 0.05% Tween 20. Cells were incubated on ice with fluorescein isothiocyanate (FITC)-labeled goat anti-mouse IgG for 30 min, washed four times with cold PBS (pH 7.2) containing 0.05% Tween 20, and then evaluated by using a fluorescence microscope.

To determine the cytopathic effect (CPE) of influenza virus in SJPL cells, confluent monolayers of SJPL in a 6-well plate (Becton Dickinson, Franklin Lakes, N.J.) were infected with A/Sydney/5/97 (H3N2) (m.o.i., 2), and DMEM containing 0.3% BSA (Life Technologies) and 1 μg/ml TPCK-treated trypsin were added to each well. CPE was determined 48 h after infection.

Flow cytometric analysis of virus receptor expression on the cell surface. The analysis was carried out with the Digoxigenin (DIG) Glycan Differentiation Kit (Roche Molecular Biochemicals, Indianapolis, Ind.) with modifications. SJPL cells, Mv1Lu cells, and MDCK cells trypsinized and resuspended at a concentration of 10⁷ cells per ml in the binding medium (Tris-buffered saline, pH 7.6; 0.5% BSA; and 1 mM Ca²⁺, 1 mM Mg²⁺, and 1 mM Mn²⁺). SJPL cells (3×10⁶) and MDCK cells (3×10⁶) were incubated for 30 min at room temperature with DIG-labeled lectins Maackia amurensis agglutinin (MAA), which specifically binds to Sia2-3Gal, or Sambucus nigra agglutinin (SNA), which specifically binds to Sia2-6Gal. Cells were washed three times with cold PBS (pH 7.2) containing 0.05% Tween 20, and FITC-labeled anti-DIG antibody diluted in PBS (pH 7.2) containing 0.5% BSA was added to the cells. After a 30-min incubation on ice, the cells were washed three times with cold PBS (pH 7.2) containing 0.05% Tween 20, and the fluorescence intensity of the cells was analyzed with a FACS Calibur Fluorospectrometer (Becton Dickinson).

Annexin-V-FL UOS staining, propidium iodide staining, and flow cytometric analysis. Approximately 5×10⁶ MDCK cells and 5×10⁶ SJPL cells were infected with A/Sydney/5/97 (H3N2) (m.o.i., 5); we collected cells by trypsinizing them 10 h after infection. Annexin-V-FLUOS staining and flow cytometric analysis were performed according to the manufacturer's instructions (Roche Molecular Biochemicals). Briefly, 1×10⁶ cells were incubated with 100 μl staining solution prepared in 1000 μl HEPES buffer added with 20 μl Annexin-V-FLUOS, and 20 μl propidium iodide for 10 min at room temperature, and the fluorescence intensity of the stained samples was analyzed with a FACS Calibur Fluorospectrometer (Becton Dickinson).

DNA fragmentation assay. Fragmentation of cellular DNA was determined as described (Ishida et al., EMBO J., 1992, 11:3887-3895, Hinshaw et al., J. Virol, 1994, 68:3667-3673) but with slight modifications. Approximately 5×10⁶ MDCK cells and 5×10⁶ SJPL cells were infected with A/Sydney/5/97 (H3N2), A/Chicken/NY/13307-3/95 (H7N2), or A/Swine/IA/17672/88 (H1N1) influenza viruses (m.o.i. 5), and cells were harvested 18 hours after infection. The harvested cells were washed in PBS and resuspended in 500 μl ice-cold lysis buffer (10 mM Tris, pH 7.5; 0.5% Triton X-100 [Sigma]) before they were incubated on ice for 30 min. The lysates were centrifuged for 10 min at 12,000×g at room temperature to remove cellular debris, and the supernatant were extracted once with buffered phenol and once with buffered phenol-chloroform. DNA in the supernatant was collected by ethanol precipitation with 3 M sodium acetate (pH 5.2). DNA was dissolved in 20 μl sterile water, treated with RNase A (Sigma), and subjected to electrophoresis through 2% agarose (GTG SeaKem agarose; FMC BioProducts, Rockland, Me.) in TAE buffer (0.04 M Tris-acetate, pH 7.2; 0.001 M EDTA). The gel was stained with ethidium bromide. A 100-bp DNA ladder (Promega Corporation, Madison, Wis.) was used as a molecular marker.

Reverse transcriptase assay. To determine whether SJPL cells express procine endogenous retrovirus, a non-radioactive reverse-transcriptase assay was performed as described by the manufacturer (Roche Molecular Biochemicals). Briefly, 10 ml of supernatant from confluent SJPL cells in 75-cm² tissue-culture flasks was collected and subjected to ultracentrifugation (100,000×g) for 30 min. Pellets were resuspended in 40 μl lysis buffer (50 mM Tris, 80 mM potassium chloride, 2.5 mM dithiothreitol [DTT], 0.75 mM EDTA, and 0.5% Triton X-100 [pH 7.8]) and incubated at room temperature for 30 min. After the pellets were completely solubilized, 20 μl of reaction mixture (46 mM Tris-HCl, 266 mM potassium chloride, 27.5 mM magnesium chloride, 9.2 mM DDT 10 μM dUTP, 10 μM dTTP, and template-primer hybrid of poly[A] and oligo[dT]) was added to tubes containing lysates. The mixtures were incubated for 10 hours at 37° C. HIV-1 reverse transcriptase (0.125 ng/well) served as a positive control. After the reverse transcriptase reaction was complete, samples (60 μl) and dilutions of HIV-1 reverse transcriptase dilution (60 μl) were transferred into the wells of the microtiter plate precoated with streptavidin and incubated for 1 h at 37° C. After the wells were washed 5 times, 200 μl of anti-DIG-peroxidase antibody was added to each well, and the plates were incubated for 1 h at 37° C. After the wells were washed 5 times, 200 ill of 2′, 2-azino-di [3-ethyl-benzthiazoline-b-sulfonic acid] substrate solution was added to each well, and the plates were incubated at room temperature for 30 min before the absorbance of the samples at 405 mm was determined.

Statistical analysis. Analyses were performed with the aid of SAS dn SPLUS programs (Venables and Repley, Modern Applied Statistics, 1997, New York:Springer) to compare replication efficiency of influenza viruses in between SJPL and MDCK cells.

### Results

Expression of influenza virus proteins and influenza virus-induced cytopathic effect in SJPL cells. We first evaluated the morphology of SJPL cells to determine whether cytopathic effects (CPE) occurred after infection with an influenza virus. MDCK cells infected with influenza viruses show marked cytopathogenicity. SJPL cells infected with 2 m.o.i of A/Sydney/5/97 (H3N2) showed strong CPE but uninfected cells showed no sign of cell damage. The mode of CPE in MDCK cells and SJPL cells infected with influenza viruses was different. Approximately 16 hours after infection, pores on the monolayer of MDCK cells started to appear, and the size of pores increased until the entire monolayer was destroyed and detached from the surface of the flask. In contrast, the apical surfaces of SJPL cells started to be destroyed at 18 hours after infection, and the area of the apical surface that was destroyed continued to increase, but some cells (20%) remained attached to the flask surface even after 72 hours.

To determine whether the newly established SJPL cell line could express structural proteins of influenza virus, we used a monoclonal antibody to nucleoprotein in an indirect immunofluorescent assay to detect the viral protein in SJPL cells that had been infected with A/Sydney/5/97 (H3N2). The results showed that the cytoplasm and nuclei of infected cells contained nucleoprotein but similar regions of uninfected cells did not.

Replication efficiencies of mammalian influenza viruses in SJPL cells. We compared the replication efficiencies of human, swine, and equine influenza viruses in SJPL cells with those in MDCK cells. Confluent monolayers of SJPL and MDCK cells were infected with 10 EID₅₀ of each virus. Viral titration was performed by using SJPL cells for viruses that had been grown in SPJL cells and MDCK cells for viruses that had been grown in MDCK cells. Most of the human viruses grew to higher titers in SJPL cells than in MDCK cells (Table 1). The titers of human influenza viruses grown in SJPL cells ranged from 2.00 to 6.50 log₁₀TCID₅₀/ml, whereas the titers of the same viruses grown in MDCK cells ranged from 0.00 to 4.25 log₁₀TCID₅₀/ml. The titers of AJBel/42 (H1N1) and A/Port Chalmers/1/73 (H3N2) viruses were at least 100-fold higher in SJPL cells than in MDCK cells; the titers of A/PR/8/34 (H1N1), A/Japan/305/57 (H2N2), A/Korea/68 (H2N2), and B/Lee/40 viruses were at least 10-fold higher in SJPL cells than in MDCK cells. Although A/Bel/42 (H1N1) did not grow to detectable levels in MDCK cells, it replicated in this cell line when higher doses of this virus were used for infection.

TABLE 1
Comparison of replication efficiencies of mammalian
influenza A and B viruses in SJPL cells with those
of the same viruses in MDCK cells

Virus titer
(log₁₀TCID₅₀/ml)^{a}

| Virus Strain | SJPL | MDCK |
|---|---|---|
| A/PR/8/34(H1N1) | 4.5, 4.0 | 3.0, 2.75 |
| A/Bel/42(H1N1) | 2.5, 2.0 | <^{b} |
| A/Japan/305/57(H2N2) | 5.0, 4.75 | 3.5, 3.0 |
| A/Korea/68 (H2N2) | 5.5, 5.0 | 4.0, 3.75 |
| A/HK/1/68 (H3N2) | 4.5, 4.0 | 3.75, 3.5 |
| A/Port Chalmers/1/73(H3N2) | 6.25, 6.5 | 4.0, 4.25 |
| B/Lee/40 | 5.5, 5.0 | 3.75, 3.5 |
| A/Swine/Ned/3/80(H1N1) | 6.0, 6.25 | 4.25, 4.0 |
| A/Swine/Guelph/41848/97(H3N2) | 6.5, 6.0 | 3.5, 3.25 |
| A/Swine/TX/4199-2/98(H3N2) | 6.0, 6.25 | 5.5, 5.0 |
| A/Swine/NC/35922/98(H3N2) | 3.75, 3.75 | 2.0, 1.75 |
| A/Equine/KY/1/81(H3N8) | 2.0, 2.0 | 2.0, 1.75 |
| A/Equine/Prague/1/56(H7N7) | 1.0, 1.0 | 1.0, 1.0 |
| A/Equine/Alaska/29759/91(H3N8) | 3.25, 3.50 | 3.0, 3.0 |
| A/Equine/London/1416173(H7N7) | 3.5, 3.25 | 3.0, 3.25 |

^{a}Values of two independent experiments are given.
^{b}The levels of virus were below the limit of detection.

Replication efficiency of swine influenza viruses was also greater in SJPL cells than in MDCK cells (Table 1). The virus titers in SJPL cells ranged from 3.75 to 6.50 log₁₀TCID₅₀/ml, whereas the titers for the same viruses in MDCK cells ranged from 1.75 to 5.50 log₁₀/TCID₅₀/ml. The titers of A/Swine/Ned/3/80 (H1N1) and A/Swine/NC/35922/98 (H3N2) viruses were approximately 100-fold higher in SJPL cells than in MDCK cells. The titers of A/Swine/Guelph/41848/97 (H3N2) viruses were approximately 1000-fold greater in SJPL cells than in MDCK cells.

The replication efficiencies of equine influenza viruses were lower than those of the swine viruses in SJPL cells and in MDCK cells. The range of titers of A/Equine/KY/1/81 (H3N8), A/Equine/Alaska/29759/91 (H3N8), A/Equine/Prague/1/56 (H7N7), and A/Equine/London/1416173 (H7N7) viruses in MDCK and SJPL cells was 1.00 to 3.50 log₁₀TCID₅₀/ml.

We assessed the difference in replication efficiencies of the mammalian viruses in the two cell lines by performing analysis of variance for the two-way layout with replicates. Sufficient evidence was found at the 0.05 level of significance to conclude that the replication efficiencies of mammalian influenza viruses are significantly higher in SJPL cells than in MDCK cells (P<0.0001).

Replication efficiencies of avian influenza viruses in SJPL cells. Avian species harbor all subtypes of influenza A viruses (Hinshaw and Webster, 1982, In Basic and Applied Influenza Research, p. 79-104, Beard (ed.), CRC press, Boca Raton, Fla.). Because avian influenza viruses are potential pandemic influenza viruses in humans, we determined the replication efficiencies of avian viruses in SJPL cells and in MDCK cells. All representative avian influenza viruses replicated in SJPL cells; the range of titers was 1.50 to 5.25 log₁₀TCID₅₀/ml. In MDCK cells, most representatives of the avian virus subtypes replicated, but the avian influenza viruses A/Mallard/Alberta/119/98 (H1N1), A/RuddyTurnstone/DE/259/98 (H9N9), A/Mallard/Alberta/223/98 (H10N8), and A/Shorebird/DE/224/97 (H13N6) did not grow in MDCK cells when the cells were inoculated with a dose of 10 EID₅₀. Higher inoculating doses of these viruses resulted in their replication in MDCK cells. The virus titers in MDCK cells ranged from 0.00 to 5.25 log₁₀TCID₅₀/ml (Table 2). A/M4allard/Alberta/205/98 (H2N9), A/Shorebird/DE/207/98 (H3N8), A/Ruddy Turnstone/DE/69/98 (H6N8), A/Shorebird/DE/11/95 (H11N9), A/M4allard/Astrakhan/263/82 (H14N5), and A/Wedgetailed Shearwater/Western Australia/2576/79 (H15N9) viruses replicated well in SJPL cells (range of titers, 3.00 to 5.25 log₁₀TCID₅₀/ml) and in MDCK cells (range of titers, 2.50 to 5.25 log₁₀TCID₅₀/ml). The replication efficiencies of avian influenza viruses in SJPL cells were higher than in MDCK cells (P<0.0001).

TABLE 2
Comparison of replication efficiencies of avian influenza viruses
in SJPL cells with those of the same viruses in MDCK cells

Virus Titer
(log₁₀TCID₅₀/ml)^{a}

| Virus Strain | SJPL | MDCK |
|---|---|---|
| A/Mallard/Alberta/119/98(H1N1) | 3.5, 3.0 | <^{b} |
| A/Mallard/Alberta/205/98(H2N9) | 4.5, 4.75 | 5.0, 5.25 |
| A/Shorebird/DE/207/98(H3N8) | 3.0, 3.0 | 2.5, 2.5 |
| A/Mallard/Alberta/47/98(H4N1) | 5.0, 5.25 | 2.25, 2.0 |
| A/Duck/Singapore/3/97(H5N3) | 4.5, 4.0 | 3.0, 3.0 |
| A/Ruddy Turnstone/DE/69/98(H6N8) | 3.75, 3.5 | 3.0, 2.75 |
| A/Chicken/NY/13307-3/95(H7N2) | 4.25, 4.5 | 2.0, 2.0 |
| A/Red Knot/DE/254/94(H8N4) | 4.5, 4.0 | 1.0, 1.0 |
| A/RuddyTurnstone/DE/259/98(H9N2) | 3.0, 2.75 | <^{b} |
| A/Mallard/Alberta/223/98(H10N8) | 3.0, 3.0 | <^{b} |
| A/Shorebird/DE/11/95(H11N9) | 5.0, 4.75 | 4.5, 4.0 |
| A/Mallard/Alberta/52/97(H12N5) | 2.0, 2.25 | 1.0, 1.0 |
| A/Shorebird/DE/224/97(H13N6) | 1.5, 1.75 | <^{b} |
| A/Mallard/Astrakhan/263/82(H14N5) | 5.0, 5.25 | 4.5, 4.25 |
| A/Wedgietailed Shearwater/ | 5.25, 5.0 | 5.0, 4.75 |
| Western Australia/2576/79(H15N9) | | |

^{a}Values of two independent experiments are given.
^{b}The levels of virus were below the limit of detection

Replication efficiencies of highly pathogenic H5N1 influenza viruses in SJPL cells. We compared the replication efficiencies of the highly pathogenic H5N1 influenza viruses in SJPL cells with those of the same viruses in MDCK cells. A/HK/156/97 (H5N1), which was originally isolated from a 3-year-old boy, replicated well in MDCK cells and in SJPL cells; the titers ranged from 5.0 to 5.25 log₁₀TCID₅₀/ml (Table 3). Unlike A/Chicken/HK/258/97, A/Chicken/HK/728/97 (H5N1) virus did not grow well in either cell line. Titers of A/Chicken/HK/728/97 (H5N1) were at least 10⁵-fold less than those of A/Chicken/IW258/97 (H5N1) in SJPL cells. The addition of 1 μg/ml of TPCK-treated trypsin to the infection media did not make a difference in viral yield in either cell line. The replication efficiencies of the highly pathogenic H5N1 viruses were significantly greater in SJPL cells than in MDCK cells (P=0.0021).

TABLE 3
Comparison of replication efficiencies of H5N1
influenza viruses in SJPL cells with those of
the same viruses in MDCK cells

| | Virus Titer (log₁₀TCID₅₀/ml)^{a} | |
|---|---|---|
| Virus Strain | SJPL | MDCK |
| A/Chicken/HK/258/97 | 6.5, 6.5(6.5) | 5.0, 4.75(4.5) |
| A/HK/156/97 | 5.0, 5.0(5.5) | 5.0, 5.25(5.0) |
| A/Goose/HK/W374/97 | 5.5, 5.0(5.5) | 5.0, 5.25(5.0) |
| A/Goose/HK/437-4/97 | 3.0, 3.25(3.5) | 2.0, 1.75(3.0) |
| A/Chicken/HK/728/97 | 1.5, 1.0(2.5) | 1.75, 2.0(1.5) |

^{a}Value of two independent experiments are given.
The values in parentheses are the virus titers after 1 μg/ml TPCK-treated trypsin was added to the infection media.

Kinetics of influenza virus replication in SJPL cells. To determine the kinetics of virus replication in SJPL cells, we compared the growth curves of representative viruses in SJPL cells with those of the same representative viruses in MDCK cells. Three influenza viruses, A/Sydney/5/97 (H3N2), B/Memphis/1/84, and A/Swine/IA/17672/88 (H1N1), were used at 20 EID₅₀. To minimize the effect that the species difference in cell lines might have on virus replication, we used SJPL cells to determine the titer of viruses that were initially grown in SJPL cells and MDCK cells to determine the titer of viruses that were initially grown in MDCK cells. The growth pattern of each virus in SJPL cells was similar to that of the same virus in MDCK cells. The time of peak viral growth in both SJPL and MDCK cells was 50 hours after infection, and after that time, the viral titers started to decline. At 72 hours after infection, the infectious viral titers were approximately 10²- to 10³-fold less than those at 50 hours after infection. In both cell lines, detectable levels of A/Swine/IA/17672/88 (H1N1) were first observed at 14 hours after infection, but detectable levels of B/Memphis/1/84 and A/Sydney/5/97 (H3N2) were not seen until 20 hours after infection. At the times of peak growth, the mean titers of B/Memphis/1/84 and A/Sydney/5/97 (H3N2) were 10-fold greater in SJPL cells than in MDCK cells, whereas the mean titer of A/Swine/Lk/17672/88 (H1N1) at the time of peak growth (i.e., 50 hours after infection) was approximately 100-fold greater in SJPL cells than in MDCK cells.

Primary influenza virus isolation from human clinical samples and antigenic stability of cultured viruses. Because some strains of human influenza viruses from clinical samples do not grow in eggs, MDCK cells are often used for the growth and isolation of influenza viruses from human clinical samples. Recently isolated human influenza viruses require adaptation to eggs before their variants can grow in eggs. We determined whether SJPL cells could be used to grow and isolate influenza viruses from clinical gargle samples. MDCK cells were used for comparison. Fourteen influenza viruses in 20 human clinical samples were grown and isolated in SJPL cells and in MDCK cells. Results of the M assays indicated that all the isolates were influenza A viruses (H3N2). The viral titers in both cell lines ranged from 2.0 to 4.5 log₁₀TCID₅₀/ml (Table 4).

TABLE 4
Primary isolation of influenza A viruses (H3N2)
from human clinical samples.

| | |
|---|---|
| | Virus Titers^{b} |
| | (log₁₀TCID₅₀/ |
| No. of HA-positive cells^{a}/ | ml) |

| Host cell | No. of clinical samples | Range | Mean |
|---|---|---|---|
| MDCK | 14/20 | 2.0-4.5 | 3.196 |
| SJPL | 14/20 | 2.0-4.5 | 3.300 |

^{a}HA tests were performed 72 h after inoculation.
^{b}MDCK cells were used to determine viral titers. The titers of all 14 virus isolates were measured.

The hallmark of influenza virus propagation in mammalian cells is that the growth of human influenza viruses in this type of cell usually does not lead to antigenic change; this result is in contrast to the antigenic changes that occur in influenza viruses cultured in eggs (Robertson et al., 1995, Virology 179:3540). To determine whether the growth of influenza A virus from clinical samples can lead to changes in amino acids of HA1, SJPL and MDCK cells were inoculated with clinical gargle samples from which A/Memphis/4/99 (H3N2) was isolated. As a control, the virus in the clinical samples was adapted to eggs. After three passages, the virus was isolated, and the gene segment encoding the HA1 region was sequenced. Critical amino acids (Lys-154, Gln-156, Lys-173, Ser-186, Leu-194, Ser-199, Arg-220, Asn-246, and Thr-248) of the HA1 in the virus from the original clinical sample were identical to those of the HA1 in the viruses passaged three times in MDCK cells and in SJPL cells (Table 5). In addition, the rest of the predicted amino acid sequences within HA1 were identical in the original virus and those grown in SJPL cells and in MDCK cells. Unlike the viruses grown in MDCK cells and in SJPL cells, influenza virus passaged in eggs contained two amino acid substitutions in the HA globular head (194L→1 and 220R→S).

TABLE 5
Comparison of critical amino acids in the HA1 region of viruses
grown in MDCK cells, in SJPL cells, and in eggs

| | Amino acid at the indicated position in HA1 | | | | | | | | |
|---|---|---|---|---|---|---|---|---|---|
| Source of virus | 154 | 156 | 173 | 186 | 194 | 199 | 220 | 246 | 248 |
| Original saliva sample | K | Q | K | S | L | S | R | N | T |
| MDCK | K | Q | K | S | L | S | R | N | T |
| SJPL | K | Q | K | S | L | S | R | N | T |
| Eggs | K | Q | K | S | I | S | S | N | T |

Human H3N2 (A/Memphis/4/99) influenza virus was passaged three times in MDCK cells, in SJPL cells, and in eggs before the nucleotide sequences were determined.

Influenza virus receptors on SJPL cells. Influenza viruses enter cells by binding to sialylglycoconjugates on the surface (Gambaryan et al., Virology, 1997, 232:345-350; Carroll et al., Virus Res., 1985, 3:165-179; Paulson et al., The Receptors, 1985, Vol. 2, pp. 131-219; Connor et al., Virology, 1994, 205:17-234; Matrosovich et al., Virology, 1997, 233:224-234). We determined whether influenza virus receptors were present on the newly established SJPL cell line. Receptor specificity was evaluated by incubating the cells with DIG-labeled lectins (Maackia amurensis agglutinin [MAA], which binds specifically to Sia2-3Gal; and Sambucus nigra agglutinin [SNA], which binds specifically to Sia2-6Gal) and FITC-labeled anti-DIG antibody and then performing flow cytometric analysis. MDCK cells and Mv1Lu cells as well as cells stained only with the antibody, but not with lectins, were used as controls. SJPL, MDCK, and Mv1Lu cells expressed Sia2-3Gal- and Sia2-6Gal-containing sialylglycoconjugates on their surface, but the numbers of receptors on the cell surface differed among the cell lines. The peak log intensity of Sia2-3Gal-containing receptors on the surface of Mv1Lu cells was 1.5, and that of Sia2-6Gal-containing receptors was 1.8, the peak log intensity of Sia2-3Gal-containing receptors on the surface of MDCK cells was 1.75, and that of Sia2-6Gal-containing receptors was 2.4, the peak log intensity of Sia2-3Gal-containing receptors on the surface of SJPL cells was 2.4, and that of Sia2-6Gal-containing receptors was 2.9. Therefore, SJPL cells have more Sia2-3Gal- and Sia2-6Gal-containing receptors than the other two cell lines.

Influenza virus-induced damage in SJPL cells and in MDCK cells. It has been reported that influenza infection of epithelial cells leads to apoptosis (Hinshaw et al, J. Virol, 1994, 68:3667-3673; Takizawa et al., J. Gen. Virol, 1993, 74:2347-2355). Using an Annexin-V binding assay, a propidium iodide staining assay, and a DNA fragmentation assay, we determined whether influenza virus infection of the newly established porcine lung epithelial cell line also causes apoptosis. Annexin-V is a Ca²⁺-dependent phospholipid-binding protein with a high affinity for phosphatidylserine that translocates from the inner side of the plasma membrane to the outer side during an early stage of apoptosis but not during necrosis (Martin et al., J. Biol. Chem., 273:43454349, Fadok et al., J. Immunol., 1992, 148:2207-2216). Propidium iodide binds to DNA of necrotic cells, which have damaged membranes, but does not bind to DNA of membrane-intact apoptotic cells. Ten hours after infection, 43% of SJPL cells infected with A/Sydney/5/97 (H3N2) influenza virus were stained with propidium iodide, but less than 1% of the population was detected with the FLUOS-conjugated annexin-V. Approximately 60% of MDCK cells infected, with A/Sydney/5/97 (H3N2) bound to annexin-V, and less than 1% were weakly stained with propidium iodide. Uninfected SJPL cells and MDCK cells did not either bind to annexin-V or stain with propidium iodide.

Eighteen hours after infection, SJPL cells infected with A/Sydney/5/97 (H3N2), A/Chicken/NY/13307-3/95 (H7N2), and A/Swine/IA/17672/88 (H1N1) (m.o.i. 5) did not show signs of DNA fragmentation, whereas MDCK cells infected with A/Sydney/5/97 (H3N2), A/Chicken/NY/13307-3/95 (H7N2), and A/Swine/IA/17672/88 (H1N1) exhibited DNA fragmentation visible upon resolution of DNA on 2% agarose gel. Uninfected SJPL cells and MDCK cells did not show signs of DNA fragmentation.

Reverse transcriptase activity. The recent progress in the field of xenotransplantation has raised concerns about the possible transmission of porcine endogenous retrovirus (PERV) to humans. Results of previous studies showed that PERV from a porcine kidney cell line (PK15) can infect many types of human cell lines (kidney, lung, muscle, and lymphoid) in vitro (Patience et al., Nat. Med., 1997, 3:282-286; Le Tissier et al., Nature, 1997, 389:681-682; Wilson et al., J. Virol, 1998, 72:3082-3087). If PERV from the SJPL cell line is present in the growth medium, then the SJPL cell line would not be a suitable candidate for use in influenza virus vaccine production. Because of this possibility of PERV contamination, we assayed the level of reverse transcriptase activity in tissue-culture supernatant of SJPL cells; with this assay, we could detect any type of retrovirus that was present. Reverse transcriptase activity was not detected in supernatant of SJPL cells, i.e., the levels of activity were similar to those of background (0.05 OD₄₅₀). In contrast, high levels of reverse transcriptase activity (1.5 OD₄₅₀) were detected in medium containing the positive control HIV-1 reverse transcriptase.

The presence of PERV in SJPL cell line was also tested using PERV-specific POL PCR primers PB905 (5′CCGCAGGGATGGGTTTGGCAAAGCA3′) and PB906 (5′ACGTACTGGAGGAGGGTCACCTGA3′) and no virus was detected either by amplification of samples derived from tissue culture supernatant or from cellular RNAs. In contrast, in a parallel amplification reaction, PERV-specific nucleic acid was detected in both supernatant and cellular RNA fractions of porcine kidney cell line PK-15 (ATCC Accession No. CCL-33). This cell line is known to constitutively express PERVs. Actin RNA, which was used as an internal control, was detected in all samples.

### Discussion

We obtained and characterized a novel spontaneously established porcine lung epithelial cell line (SJPL) and determined whether it could be used in the surveillance of influenza viruses, in the study of pathogenesis and host range of influenza viruses, and possibly in the production of more effective influenza virus vaccines. As disclosed above, we showed that this cell line productively supported the replication of representatives of all tested subtypes of avian, human, and swine influenza viruses. The efficiency of primary isolation of influenza viruses from human patients was comparable to that of MDCK cells. Passage of virus from a human clinical sample in SJPL cells did not result in amino acid changes around the receptor binding site of HA, while passage of virus from this clinical sample in eggs resulted in two amino acid changes around the receptor binding site of HA. SJPL cells expressed more Sia2-3Gal- and Sia2-6Gal-containing receptors than did MDCK or Mv1Lu cells. Necrosis, but not apoptosis, occurred in SJPL cells infected with influenza virus.

Efforts have been made previously to find cell lines that support the productive replication of influenza virus and allow the virus to maintain its original antigenicity. African green monkey kidney (Vero) cells fully support replication of influenza A and B viruses (Govorkova et al., J. Virol., 1996, 70:5519-5524), but influenza viruses have to be extensively adapted before they can be grown in this cell line. Repeated addition of trypsin to the culture medium is also needed for multicycle growth of the influenza viruses (Kaverin et al., J. Virol., 1995, 69:2700-2703). BHK cells also support replication of influenza viruses, but growth in BHK cells, like that in eggs, results in the selection of receptor-binding variants of human influenza viruses (Govorkova et al., Virology, 1999, 262:31-38). MDCK cells are currently considered to be the best cell line for supporting the growth of influenza viruses, but this cell line causes cancer in nude mice (Govorkova et al., 1996, supra). Therefore, MDCK cells have not been licensed for use in the production of vaccines. Cold-adapted influenza virus vaccine can be produced in specific pathogen-free (SPF) eggs, but in a pandemic, the supply of this type of egg will not be sufficient to meet demand.

An important advantage of SJPL cells for use in influenza vaccine production is that propagation of human influenza A viruses in these cells does not lead to antigenic changes in the HA molecule. Current vaccines prepared from human influenza viruses adapted for growth in eggs contain variants whose HA molecules differ from that of the original human virus by at least one or two amino acids (Katz et al., Virology, 1987, 156:386-395, Robertson et al., Vaccine, 1995, 13:1583-1588). This variation will result in the immune escape of influenza viruses in the immunized humans. In a mouse model of influenza virus infection, a single amino acid substitution in the HA molecule rendered a candidate vaccine for an influenza A virus (H3) ineffective (Kodihalli et al., J. Virol. 1995, 69:4888-4897). In that study, the virus with a substitution at Lys 156 in the HA molecule was poorly immunogenic and did not prevent infection by viruses that had been grown in MDCK cells. SJPL cells may be a good candidate for use in producing a better human vaccine that maintains the antigenicity of the original virus.

SJPL cells express Sia2-3Gal- and Sia2-6Gal-containing sialylglycoconjugates, which serve as receptors for influenza virus. Ito et al. (J. Virol., 1998, 72:7367-7373) reported that receptors for human and avian influenza viruses are present on the surface of pig tracheal cells and suggested that this finding may explain how pigs serve as mixing vessels in which influenza viruses that can cause pandemics are created. However, Mv1Lu and MDCK cells also express both receptors on their cell surface. It is possible that unknown cellular factors, in addition to receptors on the cell surface, are responsible for creating an environment within the pig for the generation of human-avian virus reassortants.

Replication of influenza viruses, even those of the same subtype, appears to differ in SJPL and MDCK cells. Most importantly, the viral titers produced in SJPL cells are consistently higher than titers produced in MDCK cells (Tables 1 and 2).

Our findings also indicate that influenza virus-induced damage of SJPL cells is due to necrosis rather than apoptosis. It has been shown previously that influenza infection of HeLa cells triggers the expression Fas and Fas-ligand (FasL) on the cell surface. Fas (CD95) is a cell surface receptor that transduces apoptotic signal (Wallach et al., Annu. Rev. Immunol., 1999, 17:331-367, Ashkenazi et al., Science, 1998, 281:1305-1308). After Fas binds to FasL, the death domain in the cytoplasmic portion of Fas recruits Fas-associated death domain proteins (FADD) and the caspase-8 proenzyme; these factors then participate in zip caspase activation and apoptosis (Chinnaiyan et al., Cell, 1995, 81:505-512, Kischkel et al., EMBO J., 1995, 14:5579-5588, Huang et al., Nature, 1996, 384:638-641, Martin et al., J. Biol. Chem., 1998, 273:4345-4349). It is possible that influenza virus infection of SJPL cells does not induce expression of Fas and FasL on the cell surface; this lack of expression may explain why uninfected SJPL cells do not undergo apoptosis.

The differences in the types of death observed in infected cells could also be due to differences in mitochondrial permeabilization. Mitochondria play an important role in regulating apoptosis (Green and Reed, Science, 1998, 281:1309-1312). When the mitochondrial membrane is perturbed, proapoptotic factors such as cytochrome c, procaspase-2, -3, and -9, and apoptosis-inducing factor are released from the mitochondrial membrane space and are free to participate in the degradation phase of apoptosis (Liu et al., Cell, 1996, 86:147-157, Mancini et al., J. Cell Biol., 1998, 140:1485-1495, Zamzami et al., J. Exp. Med., 1996, 183:1533-1544). The perturbation of the membrane is regulated by Bcl-2 family members. Indeed, apoptosis was shown to occur in infected MDCK cells with low levels of Bcl-2 (Hinshaw et al., J. Virol, 1994, 68:3667-3673).

The absence of apoptosis in SJPL cells may also be associated with the intracellular level of transcriptional activator nuclear factor-κB (NF-κB). Recent findings showed that the NF-κB induces the expression of factors that prevent apoptosis (Antwerp et al., 1996, Wang et al., Science, 1996, 274:784-787). We hypothesize that infection of SJPL cells by influenza virus induces the expression of NF-κB and that the subsequent expression of protective factors prevents apoptosis of infected SJPL cells. In contrast, influenza virus infection of MDCK cells may not induce the expression of NF-κB, and this lack of expression allows apoptosis to proceed.

It is not clear, however, whether influenza virus infection of respiratory epithelial cells causes apoptosis or necrosis in vivo. In a study of influenza A virus infection in human lungs (Guarner et al., Am. J. Clin. Pathol., 2000, 114:227-233), influenza A virus was predominantly found in intact and detached necrotic epithelial cells of larger bronchi. In chickens infected with A/Env/HK/437/99 (H5N1), foci of parenchymal cell necrosis occurred in the pancreas, brain, and heart and lymphocyte depletion and apoptosis occurred in the spleen, cloacal bursa, and thymus (Cauthen et al., J. Virol., 2000, 74:6592-6599). In mice infected with A/HK/483/97 (H5N1), apoptosis was detected in lung tissue 6 days after infection (Tumpey et al., J. Virol, 2000, 74:6105-6116). It remains to be determined whether apoptosis observed in chickens and mice is caused by the direct effect of virus replication or by bystander effects of immune responses such as those mediated by cytotoxic T lymphocytes or cytokines such as tumor necrosis factor (Zychlinsky et al., J. Immunol., 1991, 146:393400, Larrick and Wright, FASEB J., 1990, 4:3215-3223). However, we hypothesize that influenza virus infection results in necrosis, which causes inflammatory responses and subsequently permits the secondary bacterial infection of the respiratory tract. In addition, we hypothesize that the limited apoptosis is induced by an immune-mediated mechanism to eliminate virus from infected cells in the respiratory tract.

In conclusion, we show that SJPL cells support the efficient high titer replication of influenza viruses of different subtypes and that growth of human influenza virus in SJPL cells, unlike that in eggs, does not lead to antigenic changes in the virus. Because pigs are an important natural host of influenza viruses, this novel cell line may be useful in studying influenza virus-induced damage of respiratory epithelial cells and cytokine-induced damage of respiratory epithelial cells. In addition, this cell line is useful in virus surveillance and in making human vaccines that can provide greater protection against infection than those vaccines currently prepared in eggs.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

It is further to be understood that all values are approximate, and are provided for description.

All patents, applications, publications, test methods, literature, and other materials cited herein are hereby incorporated by reference.

What is claimed is:

1. An isolated porcine lung epithelial cell line having characteristics of porcine lung epithelial cell line SJPL, deposited with the American Type Culture Collection (ATCC) on Apr. 5, 2001, and assigned accession number PTA-3256.

2. The cell line of claim 1, which is SJPL cell line, deposited with the American Type Culture Collection (ATCC) on Apr. 5, 2001, and assigned accession number PTA-3256.

3. An isolated stable porcine lung epithelial cell line for influenza virus propagation produced by transfection or infection of a porcine lung epithelial cell line of claim 1 with the influenza virus or its derivatives.

4. A method for propagation of influenza virus in a cell culture, which method comprises culturing a porcine lung epithelial cell line of claim 1, wherein the cell line is transfected with recombinant influenza virus genomic plasmids or infected with influenza virus.

5. The method of claim 4, wherein the influenza virus is selected from the group consisting of human influenza, porcine influenza, equine influenza, and avian influenza.

6. The method of claim 4, wherein influenza virus propagation is not accompanied by antigenic changes in the HA1 region of the hemagglutinin molecule.

7. The method of claim 4, wherein the final titer of the influenza virus is at least 5×10⁷ PFU/ml.

8. A method for the preparation of influenza virus or virus-derived antigen comprising
(i) transfecting or infecting the porcine lung epithelial cell line of claim 1 with the influenza virus or its derivatives, and
(ii) isolating the propagated virus or virus-derived antigen.

9. The method of claim 8, wherein the influenza virus is selected from the group consisting of human influenza, porcine influenza, equine influenza, and avian influenza.

10. The method of claim 8, wherein influenza virus propagation in the porcine lung epithelial cell line is not accompanied by antigenic changes in the HA1 region of the hemagglutinin molecule.

11. The method of claim 8, wherein the final titer of the influenza virus is at least 3×10⁷ PFU/ml.

12. The method of claim 8, wherein the virus or virus-derived antigen is isolated from the cell culture medium.

13. An influenza virus produced according to the method of claim 8.

14. The influenza virus of claim 13, wherein the virus is an infectious virus.

15. The influenza virus of claim 13, wherein the virus is an attenuated virus.

16. The influenza virus of claim 13, wherein the virus is a non-infectious virus.

17. The influenza virus of claim 13, wherein the influenza virus is selected from the group consisting of human influenza, porcine influenza, equine influenza, and avian influenza.

18. An influenza virus-derived antigen produced according to the method of claim 8.

19. A vaccine composition comprising the influenza virus of claim 13 in a pharmaceutically acceptable carrier or diluent.

20. The vaccine composition of claim 19 further comprising an adjuvant.

21. The vaccine composition of claim 19, wherein the composition is free of any detectable non-influenza-derived genetic material.

22. A vaccine composition comprising the influenza virus-derived antigen of claim 18 in a pharmaceutically acceptable carrier or diluent.

23. The vaccine composition of claim 22 further comprising an adjuvant.

24. The vaccine composition of claim 22, wherein the composition is free of any detectable non-influenza-derived genetic material.

25. A method for preventing an influenza infection in an animal comprising administering to the animal the vaccine composition of claim 19 or 22.

26. The method of claim 25, wherein the animal is mammal.

27. The method of claim 26, wherein the mammal is human.

28. The method of claim 26, wherein the mammal is selected from the group consisting of porcine and equine.

29. The method of claim 25, wherein the animal is poultry.

30. A method for screening for anti-influenza therapeutics, which method comprises administering a candidate agent to the cell line of claim 1 and testing for change in the level of influenza replication or influenza-associated protein expression compared to the control untreated cell line.
